(19) United States
(12) Patent Application Publication
(10) Pub. No.: US 2024/0174962 A1
(43) Pub. Date: May 30, 2024

(54) FUNCTIONAL MEMBRANE AND MICROFLUIDIC CHIP COMPRISING SAME AND METHOD FOR MANUFACTURING SAME

(71) Applicant: UNIST(ULSAN NATIONAL INSTITUTE OF SCIENCE AND TECHNOLOGY), Ulsan (KR)

(72) Inventors: Joo Hun Kang, Ulsan (KR); Brian Choi, Ulsan (KR); Jeongwon Choi, Ulsan (KR); Tae-eun Park, Ulsan (KR)

(73) Assignee: UNIST(ULSAN NATIONAL INSTITUTE OF SCIENCE AND TECHNOLOGY), Ulsan (KR)

(21) Appl. No.: 18/551,788

(22) PCT Filed: Mar. 24, 2022
(86) PCT No.: PCT/KR2022/004103

§ 371 Date: Nov. 28, 2023

(30) Foreign Application Priority Data
Mar. 24, 2021 (KR) 10-2021-0038183

(51) Int. Cl. C12M 1/00; C12M 1/26; C12M 3/06

(52) U.S. Cl. CPC C12M 29/04; C12M 23/16; C12M 23/20; C12M 33/04; C12M 33/14

(57) ABSTRACT

The disclosure relates to a functional membrane, a microfluidic chip including the same, and a method of manufacturing the microfluidic chip. The functional membrane according to an embodiment includes a membrane having one or more pores, and a coating material covering the pores, on at least one surface of the membrane.

### TECHNICAL FIELD

The disclosure relates to a functional membrane, a microfluidic chip including the same, and a method of manufacturing the microfluidic chip.

### BACKGROUND ART

Recently, as the biotechnology field has rapidly developed and the public has started to become interested in animal ethics, the field of organ-on-a-chip simulating particular tissues or organs of humans or animals has attracted attention. An organ-on-a-chip simulates a microenvironment within a body so that interactions between various cells constituting particular tissues or organs can be observed, thereby becoming an in-vitro model allowing experiments such as drug testing or tissue regeneration to be performed. Through such an organ-on-a-chip, various cells that are simulated to have similar structures to organs or tissues within an actual body, and various physical and chemical changes that occur in the actual body can be observed in vitro.

An organ-on-a-chip may have various shapes, sizes, or the like according to an object to be simulated or the purpose of an experiment. For example, an organ-on-a-chip including a multi-layered structure in which cell culture spaces are arranged in a height direction may include thin membranes between a plurality of cell culture spaces to divide the plurality of cell culture spaces.

In the case of the above-described membranes, membranes having pores of various sizes are currently commercially available, and when an organ-on-a-chip is produced by using commercially available membranes, the organ-on-a-chip has great advantages when considering manufacturing yield, efficiency, unit cost, and convenience.

However, when such a commercially available membrane is used, during an initial cell culture process, cells may unintentionally move to other culture spaces through pores formed in the membrane that divides the cell culture spaces, and thus it is difficult to culture different cells in a completely separated state. Accordingly, it is difficult to produce tissues made up of different cells but in an attached state, such as blood vessels or surrounding tissues.

On the contrary, when the size of pores formed in a membrane is reduced to prevent cells from unintentionally moving, a cell movement phenomenon through stimulation involved in cell movement cannot be observed in an organ-on-a-chip in which cell culture has been completed, and light emitted from an optical device for image capture scatters while passing through the membrane with small pores, and thus light transmittance decreases. Accordingly, it is difficult to observe cells cultured on a membrane surface under a microscope.

To solve this problem, attempts on manufacturing a membrane by using silicon have been made, but a membrane manufactured with SiO₂ has weak mechanical strength, and thus it is difficult to use the membrane in general cell culture, and mass production thereof is not easy. In particular, a membrane made of SiO₂ is thin and has flexible characteristics, the surface thereof is easily crumpled and wrinkled in a process of adhering to a microfluidic chip, and it is difficult to perform cell alignment and form a multi-layered structure in a cell culture stage.

The above background technology is technical information that the inventors had to derive the disclosure or obtained during the process of deriving the disclosure and cannot necessarily be said to be known technology disclosed to the general public before filing of the application for the disclosure.

### DISCLOSURE

### Technical Problem

According to an embodiment, provided are a membrane, a microfluidic chip including the membrane, and a method of manufacturing the microfluidic chip, wherein the membrane has pores of sufficient size to prevent light from scattering and also prevents cells from unintentionally moving through the pores during an initial cell culture stage.

However, these objectives are illustrative, and objectives of the disclosure are not limited thereto.

### Technical Solution

A method of manufacturing a microfluidic chip according to an embodiment includes preparing substrates in which culture grooves are patterned, preparing a membrane having pores, arranging the membrane on the substrates, arranging, on at least one surface of the membrane, a coating material, covering the pores, and repeatedly arranging a coating material on the membrane covered with the coating material.

In the method of manufacturing the microfluidic chip according to an embodiment, the arranging of the coating material may further include injecting the coating material into the culture grooves, reacting and gelling the injected coating material for a certain period of time, and drying the gelled coating material for a certain period of time.

In the method of manufacturing the microfluidic chip according to an embodiment, the drying of the coating material may include drying the coating material to condense and coat the coating material on the membrane.

In the method of manufacturing the microfluidic chip according to an embodiment, the arranging of the coating material may further include, after the injecting of the coating material into the culture grooves, checking whether bubbles have been generated inside the culture grooves and removing the generated bubbles.

In the method of manufacturing the microfluidic chip according to an embodiment, the coating material may use materials having various phases. For example, as the coating material, a material in a sol state in which solid particles are dispersed in a liquid and a material in a gel state (e.g., a hydrogel), which has a certain shape in a solid or semi-solid state by gelation of the sol, may all be used. In particular, the coating material may be a hydrogel and a material in a sol state in a pre-hydrogel stage.

For example, a composition of the coating material may include and use an ECM-based biomaterial, such as collagen, gelatin, laminin, elastin, proteoglycan, glycosaminoglycan, albumin, vitronectin, heparin, heparan sulfate, hyaluronic acid, chondroitin sulfate, dermatan sulfate, keratan sulfate, tenascin, fibronectin, fibrinogen, and fibrin, and one or more of subgroups thereof. However, the disclosure is not limited thereto. A coating material may include one or more polysaccharide-based materials, such as starch, alginic acid, agar, agarose, carrageenan, cellulose, carboxymethylcellulose, chitosan, chitin, dextran, dextran salts, fluoran, pectin, keratin, silk fibroin, silk sericin, silica, lignin, guar gum, xanthan gum, gum arabic, locust bean gum, natto gum, galactomannan, glucomannan, gellan gum, or other naturally derived polymers, or a hydrogel based on a synthetic polymer including a copolymer with a hydrophilic group, such as polyacrylic acid (PAA), polyacrylonitrile (PAN), polyacrylamide (PAM), polyamidoamine (PAMAM), polymethacrylic acid (PMAA), polymethyl methacrylate (PMMA), polybutylene succinate (PBS), polycarbonate (PC), polycaprolactone (PCL), polydopamine (PDA), polydiethylaminoethyl methacrylate (PDEAEMA), polydiallyldimethylammonium chloride (PDADMAC), polyethylene (PE), polyethylene terephthalate (PET), polyethylene glycol (PEG), polyethylene oxide (PEO), polyethyleneimine (PEI), polyethylene glycol methacrylate (PEGMA), polyethylene glycol dimethacrylate (PEGDMA), polyglycerol (PG), polyglycolic acid (PGA), polyhydroxyalkanoate (PHA), polyhydroxybutyrate (PHB), Polyhydroxyethyl methacrylate (PHEMA), polylactic acid (PLA), polylactic coglycolic acid (PLGA), poly(N-isopropylacrylamide (PNIPA), polyphosphazene (PPHO), polyphosphoester (PPE), polypropylene oxide (PPO), polyurethane (PU), polyvinyl alcohol (PVA), polyvinylpyrrolidone (PVP), polysiloxane, poloxamer, poloxamine, or the like, or a sol-state material in the pre-hydrogel stage, and a mixture of one or more of the above materials may be used. In addition to the above compositions, a coating material composition may include all types of synthetic and natural compound-based hydrogels, which may condense after gelation or condense in a non-gel state due to external stimuli and factors, such as temperature and external force, and a sol-state material in the pre-hydrogel stage.

Also, the coating material composition may be a mixture of one or more of the above compositions.

In the method of manufacturing the microfluidic chip according to an embodiment, the coating material may be collagen.

The method of manufacturing the microfluidic chip according to an embodiment may further include, before the arranging of the coating material, performing blocking or plasma treatment on the microfluidic chip including the substrate and the membrane.

In the method of manufacturing the microfluidic chip according to an embodiment, solutions used for blocking treatment may be serum, bovine serum albumin (BSA), nonfat dry milk (NFDM), gelatin, casein or polymer-based polyethylene glycol (PEG), polyvinyl alcohol (PVA), and polyvinylpyrrolidone (PVP), or a mixture solution thereof.

However, the solutions use for blocking treatment are not limited thereto and may include all solutions and mixed solutions that may improve the hydrophilicity and surface energy of a surface of an activated polymer material.

In the method of manufacturing the microfluidic chip according to an embodiment, the coating material may be BSA.

In an embodiment, a solution used for blocking treatment and a coating material may be different materials. For example, when gelatin, PEG, PVA, and PVP is used as a blocking solution, the coating material may include a material other than the above materials.

In the In the method of manufacturing the microfluidic chip according to an embodiment, a gas used for plasma treatment may be, for example, oxygen (O₂), argon (Ar), nitrogen (N₂), hydrogen (H₂), or a mixture gas thereof.

In the In the method of manufacturing the microfluidic chip according to an embodiment, the performing of the plasma treatment may include performing a primary plasma treatment with an organosiloxane gas (e.g., tetramethyldisiloxane (TMDSO), hexamethyldisiloxane (HMDSO), tetraethylorthosilicate (TEOS), and hexamethylcyclotrisiloxane (HMCTSO)) and then performing a secondary plasma treatment with the above gas.

However, the gas used for plasma treatment is not limited thereto and may include all gases and mixed gases that may improve the hydrophilicity and adhesiveness of a surface of a polymer material when activated by plasma.

In the method of manufacturing the microfluidic chip according to an embodiment, the performing of the blocking treatment may include injecting a blocking solution into the culture grooves to react for a certain period of time, and then absorbing the blocking solution.

In the method of manufacturing the microfluidic chip according to an embodiment, the performing of the plasma treatment may include, for example, performing plasma treatment on the microfluidic chip including the substrate and the membrane for a certain period of time to increase the surface energy of an inner portion of the microfluidic chip and the membrane.

In the method of manufacturing the microfluidic chip according to an embodiment, a pore size formed in the membrane may be 1 nm to 50000 □.

In the method of manufacturing the microfluidic chip according to an embodiment, the substrate may be a polymethyl methacrylate (PMMA) substrate, and the membrane may be a polyethylene terephthalate (PETE) membrane.

A microfluidic chip according to an embodiment includes a first substrate in which a first culture groove is patterned, a second substrate arranged to face the first substrate and in which a second culture groove is patterned to face the first culture groove, a membrane arranged between the first substrate and the second substrate to divide a first culture channel and a second culture channel and having one or more pores, and a coating material arranged on at least one surface of the membrane to cover the pores.

In the microfluidic chip according to an embodiment, cells injected into the microfluidic chip are cultured dividedly in the first culture channel and the second culture channel with the membrane therebetween and then decompose the coating material according to a stimulation involved in cell migration, including concentration gradients of cell attractants, artificial immunity, and inflammatory reactions, or move to neighboring culture channels through the pores by passing through the coating material.

The method of manufacturing the microfluidic chip according to another embodiment may further include, before the arranging of the coating material, performing pretreatment on all or part of the membrane to have a higher surface energy than the surface energy of the substrates or the culture grooves of the substrates.

In the method of manufacturing the microfluidic chip according to another embodiment, the membrane may include a material having greater hydrophilicity than the substrates.

In addition, in the method of manufacturing the microfluidic chip according to another embodiment, the membrane may include a material that may become more hydrophilic than the substrate in a surface energy pretreatment process.

In the method of manufacturing the microfluidic chip according to another embodiment, the performing of the pretreatment may include injecting a blocking solution into the culture grooves to react for a certain period of time, and then absorbing the blocking solution.

In the method of manufacturing the microfluidic chip according to another embodiment, the f performing of the pretreatment may include treating inner portions of the culture grooves with plasma gas. In a microfluidic chip according to another embodiment, a pore size formed in the membrane may be 1 nm to 50000 □.

In the microfluidic chip according to another embodiment, pretreatment may be performed on the membrane to have a higher surface energy that the surface energy of the first substrate and the second substrate.

In the microfluidic chip according to another embodiment, the membrane may be treated with a blocking solution to have a higher surface energy than the surface energy of the first substrate, the second substrate, the first culture groove, and the second culture groove.

In the microfluidic chip according to another embodiment, the membrane may be treated with plasma gas to have a higher surface energy than the surface energy of the first substrate, the second substrate, the first culture groove, and the second culture groove.

Other aspects, features, and advantages other than those described above will become apparent from the detailed description, claims, and drawings for implementing the disclosure below.

### Advantageous Effects

In a functional membrane and a method of manufacturing a microfluidic chip including the functional membrane according to an embodiment, an unit capable of preventing unintentional movement between cells in an initial culture stage and efficiently simulating an actual biological environment by enabling movement between cells under particular conditions after the initial culture stage, while having excellent optical characteristics by providing pores of sufficient size in a membrane, which one of components of the chip, may be provided.

In the functional membrane, the method of manufacturing the microfluidic chip including the functional membrane, and the microfluidic chip according to an embodiment, unintentional movement between cells may be prevented regardless the pore size of the membrane to dividedly culture different cells in different culture channels. In addition, in the method of manufacturing the microfluidic chip and the microfluidic chip according to an embodiment, after cells are cultured and a cell layer is formed, the cells may move to neighboring culture channels according to stimulation involved in cell migration, including concentration gradients of cell attractants, artificial immunity, and inflammatory reactions, and thus cell movement or resulting immune, inflammatory response, and tissue formation as in actual tissues may be easily observed.

### DESCRIPTION OF DRAWINGS

FIGS. 1 and 2 show a microfluidic chip according to an embodiment;

FIG. 3 shows a cross section of the microfluidic chip taken along a line III-III in FIG. 2;

FIGS. 4 to 14 show a method of manufacturing a microfluidic chip according to another embodiment;

FIGS. 15 and 16 show a microfluidic chip according to a comparative example;

FIGS. 17A to 17D show nanofiber structures according to concentrations of an extracellular matrix (ECM) hydrogel of a membrane;

FIG. 18A shows a 2-dimensional (2D) image of a GFP-HUVEC cell layer in a microfluidic chip; FIG. 18B shows a 3-dimensional (3D) image of GFP-HUVEC and A549 layers in a microfluidic chip;

FIG. 19A shows an image of a vascular endothelial cell layer (blood-brain barrier) over time after culturing a microfluidic chip; FIG. 19B shows an image of a vascular endothelial cell marker protein of a microfluidic chip obtained by using an immunofluorescence staining method;

FIG. 20 shows transmittance of a blood-brain barrier formed within a channel of a microfluidic chip;

FIG. 21A shows a 3D image of a cell layer of vascular endothelial cells and glial cells; FIG. 21B shows a comparison of mRNA expression of blood-brain barrier function genes between microfluidic chip models;

FIG. 22A shows a 3D image of a vascular endothelial cell layer and non-metastatic breast cancer cells (MCF-7); and FIG. 22B shows a 3D image of a vascular endothelial cell layer and metastatic breast cancer cells (MDA-MB-231).

### BEST MODE

A method of manufacturing a microfluidic chip according to an embodiment includes preparing substrates in which culture grooves are patterned, preparing a membrane having pores, arranging the membrane on the substrates, arranging, on at least one surface of the membrane, a coating material covering the pores, and repeatedly arranging a coating material on the membrane covered with the coating material.

### Mode for Invention

As the disclosure allows for various changes and numerous embodiments, example embodiments will be illustrated in the drawings and described in detail in the written description. However, this is not intended to limit the disclosure to particular modes of practice, and it is to be appreciated that all changes, equivalents, and substitutes that do not depart from the spirit and technical scope are encompassed in the disclosure. In the description of the disclosure, like reference numerals in the drawings denote like elements even when the elements are shown in different embodiments.

Hereinafter, the disclosure will be described in detail by explaining embodiments of the disclosure with reference to the accompanying drawings, and like reference numerals in the drawings denote like elements, and thus their description will be omitted.

In the following embodiments, while such terms as “first,” “second,” etc., may be used to distinguish one component from another, and such components must not be limited to the above terms.

In the following embodiments, an expression used in the singular encompasses the expression of the plural, unless it has a clearly different meaning in the context.

In the following embodiments, it is to be understood that the terms such as “including,” “having,” and “comprising” are intended to indicate the existence of the features or components disclosed in the specification and are not intended to preclude the possibility that one or more other features or components may be added.

Sizes of components in the drawings may be exaggerated or reduced for convenience of explanation. In other words, since sizes and thicknesses of components in the drawings are arbitrarily illustrated for convenience of explanation, the following embodiments are not limited thereto.

In the following embodiments, the x-axis, the y-axis and the z-axis are not limited to three axes of the rectangular coordinate system and may be interpreted in a broader sense. For example, the x-axis, the y-axis, and the z-axis may be perpendicular to one another, or may represent different directions that are not perpendicular to one another.

When a certain embodiment may be implemented differently, a specific process order may be performed differently from the described order. For example, two consecutively described processes may be performed substantially at the same time or performed in an order opposite to the described order.

The terms used in the present specification are merely used to describe particular embodiments and are not intended to limit the disclosure. In the present specification, it is to be understood that the terms such as “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

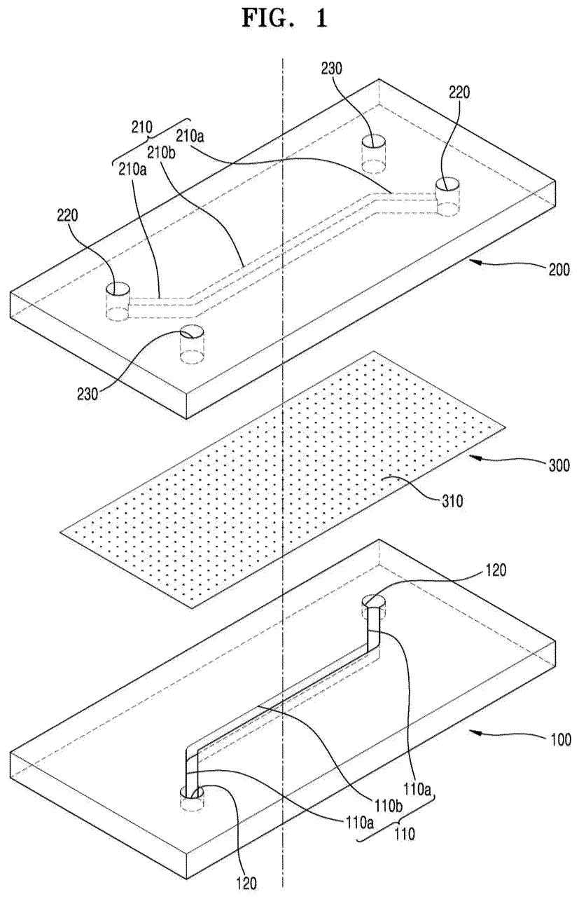

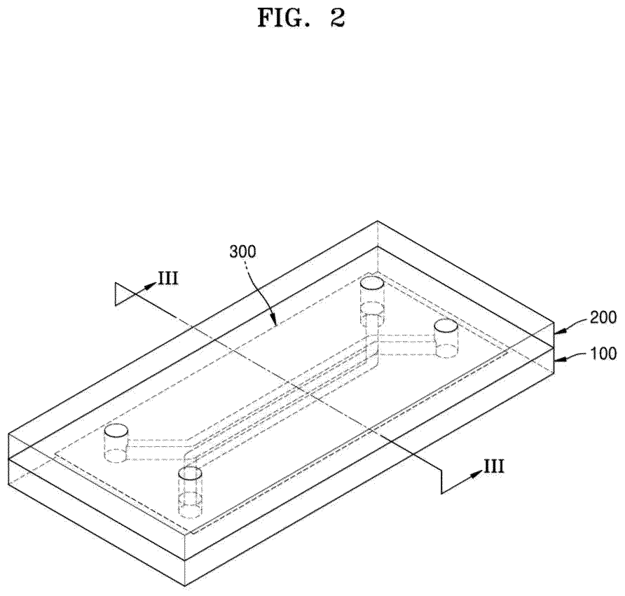

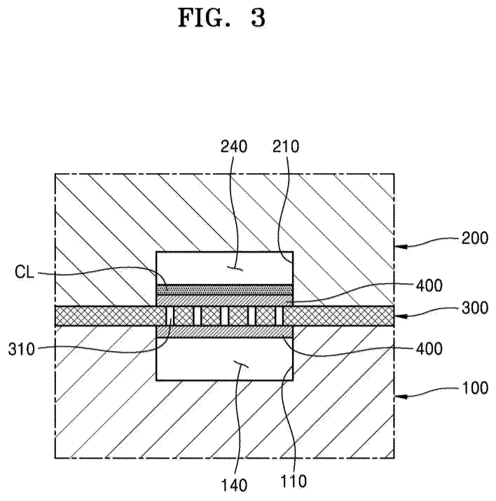

FIGS. 1 and 2 show a microfluidic chip 10 according to an embodiment, and FIG. 3 shows a cross section of the microfluidic chip 10 taken along a line of III-III FIG. 2.

Referring to FIGS. 1 to 3, the microfluidic chip 10 according to an embodiment is a cell culture device that may be used to simulate an organic of a human body or perform drug experiments on cultured cells. In an embodiment, the microfluidic chip 10 includes one or more substrates and may include a multi-layered structure including one or more culture grooves in a height direction.

The microfluidic chip 10 according to an embodiment may include a first substrate 100, a second substrate 200, a membrane 300 having one or more pores, and a coating material 400.

The first substrate 100 may be arranged on one side of the microfluidic chip 10 and have a first culture groove 110. For example, as shown in FIGS. 1 to 3, the first substrate 100 may be arranged on a lower portion of the microfluidic chip 10 and have the first culture groove 110 concavely formed on an upper surface. The first culture groove 110 is a groove formed by cutting a portion of the upper surface of the first substrate 100, wherein cells or solutions may be introduced into the first culture groove 110.

A material of the substrate is not particularly limited and may be a polymer, silicon, a metal, or a composite thereof.

In an embodiment, the first culture groove 110 may include a first inlet portion 110a and a first culture portion 110b. In more particular, as shown in FIGS. 1 and 2, the first inlet portion 110a may be arranged at each of both end portions of the first culture groove 110 and connected to a first injection port 120 to be described below. The first culture portion 110b may be arranged to be connected to each first inlet portion 110a. Accordingly, a solution and/or cell introduced from the first injection port 120 may be introduced into the first culture portion 110b by passing through the first inlet portion 110a. Also, the cell may be cultured in the first culture portion 110b. However, the disclosure is not limited thereto, and the cell may also be cultured in the first inlet portion 110a.

In another embodiment, the first culture groove 110 may not include the first inlet portion 110a, and the first culture portion 110b may directly extend from the first injection port 120. FIGS. 1 and 2 show that the first inlet portions 110a extend diagonally from respective first injection ports 120, and the first culture portion 110b is arranged between the first inlet portions 110a to be parallel to the first substrate 100, but the disclosure is not limited thereto.

In an embodiment, the first inlet portion 110a and the first culture portion 110b may at least partially be curved. Also, the first inlet portion 110a and the first culture portion 110b may at least partially be inclined. For example, each first inlet portion 110a may be inclined downward toward the first culture portion 110b. Accordingly, a solution and/or cell introduced from the first injection port 120 may be easily introduced into the first culture portion 110b. In an embodiment, in the microfluidic chip 10, the membrane 300 may be pretreated to have a higher surface energy than that of the first substrate 100. Alternatively, the membrane 300 may include a material having greater hydrophilicity than that of the first substrate 100.

In more particular, after a pretreatment (e.g., pretreatment using a blocking solution or pretreatment using plasma gas) process to be described below is undergone, the membrane 300 may be pretreated to have a higher surface energy than that of the first substrate 100 (e.g., the first culture groove 110). For example, the membrane 300 may include a material that may become more hydrophilic than the first substrate 100 after pretreatment.

Accordingly, after the coating material 400 is arranged on the membrane 300, the coating material 400 may not move to the first culture groove 110 of the first substrate 100 and may be led to the membrane 300 having higher hydrophilicity and surface energy.

A method of performing pretreatment on the first substrate 100 is not particularly limited. For example, the microfluidic chip 10 including the first substrate 100 and the membrane 300 may be plasma-treated so that a surface energy of the membrane 300 therein is greater than a surface energy of the first substrate 100 therein. Here, a gas used in plasma treatment may be a gas such as oxygen (O₂), argon (Ar), nitrogen (N₂), or the like, synthetic air (e.g., e.g. 80% nitrogen (N₂)+20% oxygen (O₂)), or a mixed gas of argon and hydrogen (e.g., 90% argon (Ar)+10% hydrogen (H₂)), or the like. However, an optimal gas mixture for plasma treatment is not limited to the above gases because the optimal gas mixture varies depending on temperature and external conditions and may include all gases and mixed gases that may improve hydrophilicity and adhesiveness of the surface of a polymer material when activated by plasma. In addition, a material for which surface treatment is desired is firstly plasma treated with organosiloxane gases (but not limited thereto), such as tetramethyldisiloxane (TMDSO), hexamethyldisiloxane (HMDSO), tetraethylorthosilicate (TEOS), and hexamethylcyclotrisiloxane (HMCTSO), and then is additionally plasma treated with gases such as oxygen, argon, nitrogen, or the like to further improve the hydrophilicity and adhesiveness of the material surface.

In an embodiment, the first culture groove 110 may be in a state in which at least a portion of an inner surface thereof is treated with blocking. In more particular, a bottom surface and a side surface of the first culture groove 110 may be treated with a blocking solution (e.g., bovine serum albumin (BSA)), and thus the coating material 400 to be described below may not be coated to the inner surface of the first culture groove 110. Accordingly, cells may not be cultured on the inner surface of the first culture groove 110 without the coating material 400, and cells may be cultured at a desired position.

In addition, various methods of leading the coating material 400 to the membrane 300 by using a surface energy difference may be used. That is, various methods may be used to perform pretreatment on at least a portion of the membrane 300 to have a greater surface energy than that of the first substrate 100.

Accordingly, the coating material 400 may be coated only on the upper surface and/or the lower surface of the membrane 300, so that cells may be aligned with the coating material 400 coated on the membrane 300 to be cultured without being cultured on the inner surface of the first culture groove 110.

In an embodiment, at least one first injection port 120 may be formed on one side of the first substrate 100. The first injection port 120 may be arranged on the upper surface of the first substrate 100 and arranged on each of both ends of the first culture groove 110. The first injection port 120 may be connected to at least a portion of a connection hole 230 of the second substrate 200 to be described below. Various solutions or cells introduced through the connection hole 230 may be introduced into the first culture groove 110 through the first injection port 120.

The second substrate 200 may be arranged on another side of the microfluidic chip 10 and have a second culture groove 210. For example, as shown in FIGS. 1 to 3, the second substrate 200 may be arranged on an upper portion of the microfluidic chip 10 and have the second culture groove 210 concavely formed on a lower surface. The second culture groove 210 is a groove formed by cutting a portion of the lower surface of the second substrate 200, wherein cells or solutions may be introduced into the second culture groove 210.

In an embodiment, the second substrate 200 may be arranged to face the first substrate 100. That is, the second substrate 200 may be arranged so that the lower surface thereof is in contact with the upper surface of the first substrate 100 with the membrane 300 therebetween. Accordingly, the first culture groove 110 may be arranged to face the second culture groove 210.

In an embodiment, the second culture groove 210 may include a second inlet portion 210a and a second culture portion 210b. In more particular, as shown in FIGS. 1 and 2, the second inlet portion 210a may be arranged at each of both end portions of the second culture groove 210 and connected to a second injection portion 220 to be described below. The second culture portion 210b may be arranged to be connected to each second inlet portion 210a. Accordingly, a solution and/or cell introduced from the second injection portion 220 may be introduced into the second culture portion 210b by passing through the second inlet portion 210a. Also, the cell may be cultured in the second culture portion 210b. However, the disclosure is not limited thereto, and the cell may also be cultured in the second inlet portion 210a.

In another embodiment, the second culture groove 210 may not include the second inlet portion 210a, and the second culture portion 210b may directly extend from the second injection portion 220.

FIGS. 1 and 2 show that the second inlet portions 210a extend diagonally from respective second injection portions 220, and the second culture portion 210b is arranged between the second inlet portions 210a to be parallel to the second substrate 200, but the disclosure is not limited thereto.

In an embodiment, the second inlet portion 210a and the second culture portion 210b may at least partially be curved. Also, the second inlet portion 210a and the second culture portion 210b may at least partially be inclined. For example, each second inlet portion 210a may be inclined downward toward the second culture portion 210b. Accordingly, a solution and/or cell introduced from the second injection portion 220 may be easily introduced into the second culture portion 210b.

In an embodiment, the second culture groove 210 may be in a state in which at least a portion of an inner surface thereof is treated with blocking. In more particular, a bottom surface and a side surface of the second culture groove 210 may be treated with a blocking solution, and thus the coating material 400 to be described below may not be coated to the inner surface of the second culture groove 210.

In an embodiment, in the microfluidic chip 10, the membrane 300 may be pretreated to have a higher surface energy than that of the second substrate 200. Alternatively, the membrane 300 may include a material having greater hydrophilicity than that of the second substrate 200.

In more particular, after pretreatment (e.g., pretreatment using a blocking solution or pretreatment using plasma gas) process to be described below is undergone, the membrane 300 may be pretreated to have a higher surface energy than that of the second substrate 200 (e.g., the second culture groove 210). For example, the membrane 300 may include a material that may become more hydrophilic than the second substrate 200 after pretreatment.

Accordingly, after the coating material 400 is arranged on the membrane 300, the coating material 400 may not move to the second culture groove 210 of the second substrate 200 and may be led to the membrane 300 having higher hydrophilicity and surface energy.

A method of performing pretreatment on the second substrate 200 is not particularly limited. For example, the microfluidic chip 10 including the second substrate 200 and the membrane 300 may be plasma-treated so that a surface energy of the membrane 300 therein is greater than a surface energy of the second substrate 200 therein. Here, a gas used in plasma treatment may be a gas such as oxygen (O₂), argon (Ar), nitrogen (N₂), or the like, synthetic air (e.g., e.g. 80% nitrogen (N₂)+20% oxygen (O₂)), or a mixed gas of argon and hydrogen (e.g., 90% argon (Ar)+10% hydrogen (H₂)), or the like. However, an optimal gas mixture for plasma treatment is not limited to the above gases because the optimal gas mixture varies depending on temperature and external conditions and may include all gases and mixed gases that may improve hydrophilicity and adhesiveness of the surface of a polymer material when activated by plasma. In addition, a material for which surface treatment is desired is firstly plasma treated with organosiloxane gases (but not limited thereto), such as TMDSO, HMDSO, TEOS, and HMCTSO, and then is additionally plasma treated with gases such as oxygen, argon, nitrogen, or the like to further improve the hydrophilicity and adhesiveness of the material surface.

In an embodiment, the second culture groove 210 may be in a state in which at least a portion of an inner surface thereof is treated with blocking. In more particular, a bottom surface and a side surface of the second culture groove 210 may be treated with a blocking solution (e.g., BSA), and thus the coating material 400 to be described below may not be coated to the inner surface of the second culture groove 210. Accordingly, cells may not be cultured on the inner surface of the second culture groove 210 without the coating material 400, and cells may be cultured at a desired position.

In addition, various methods of leading the coating material 400 to the membrane 300 by using a surface energy difference may be used. That is, various methods may be used to perform pretreatment on at least a portion of the membrane 300 to have a greater surface energy than that of the second substrate 200.

Accordingly, the coating material 400 may be coated only on the upper surface and/or the lower surface of the membrane 300, so that cells may be aligned with the coating material 400 coated on the membrane 300 to be cultured without being cultured on the inner surface of the second culture groove 210.

In an embodiment, at least one second injection portion 220 may be formed on one side of the second substrate 200. The second injection portion 220 may be arranged on the upper surface of the second substrate 200 and arranged on each of both ends of the second culture groove 210. Various solutions or cells introduced through the second injection portion 220 may be introduced into the second culture groove 210.

In an embodiment, at least one connection hole 230 may be formed on another side of the second substrate 200. The connection hole 230 may be arranged on the upper surface of the second substrate 200 and may be arranged in a position corresponding to that of the first injection port 120 of the first substrate 100. The connection holes 230 may be connected to respective first injection ports 120, and accordingly, various solutions or cells introduced through the connection hole 230 may be introduced into the first culture groove 110 through the first injection port 120.

The materials, sizes, and types of the first substrate 100 and the second substrate 200 are not particularly limited. For example, the first substrate 100 and the second substrate 200 may each have a rectangular parallelepiped shape.

The membrane 300 may be arranged between the first substrate 100 and the second substrate 200 to divide a first culture channel 140 and a second culture channel 240. For example, as shown in FIGS. 1 to 3, the membrane 300 is a plate-shaped member with pores 310 formed therein, which is inserted between the first substrate 100 and the second substrate 200. Accordingly, the first culture groove 110 formed in the first substrate 100 and the second culture groove 210 formed in the second substrate 200 may be covered with the membrane 300, and thus the first culture channel 140 and the second culture channel 240, in which cells are cultured, may be divided.

In an embodiment, the membrane 300 may be integrally formed with the first substrate 100 and the second substrate 200, or may be individually manufactured and separately coupled to the first substrate 100 and the second substrate 200. For example, the membrane 300 may include a material different from the materials of the first substrate 100 and the second substrate 200 and may be coupled to the first substrate 100 and the second substrate 200 through an adhesive and heat treatment or the like.

A pore 310 is a through hole formed in the membrane 300 in a height direction, which may be a passage through which cells cultured in the first culture channel 140 and/or the second culture channel 240 move. In an embodiment, a diameter of the pore 310 may be 1 nm to 50000 □. More preferably, the diameter of the pore 310 may be 10 Accordingly, after cell culture, a sufficient diameter of the pore 310 capable of smoothly inducing cell movement through stimulation involved in cell movement is secured, and light is prevented from being scatted by the pores 310 to some extent, and thus, cells may be easily observed through a microscope or the like.

A material, thickness, porosity (density of pores), size, and type of the membrane 300 are not particularly limited. For example, the material of the membrane 300 may be polymer, silicon, metal, or a composite thereof. In an embodiment, the membrane 300 may include a material that is more hydrophilic than the materials of the first substrate 100 and the second substrate 200.

Accordingly, as described above, when a blocking solution is injected into the first culture groove 110 and the second culture groove 210, even when the blocking solution is coated on the membrane 300 as well as the first culture groove 110 and the second culture groove 210, the coating material 400 to be described below may be coated on the membrane 300 having greater hydrophilicity and surface energy. The coating material 400 may be arranged on at least one surface of the membrane 300 to prevent cells from unintentionally moving through the pores 310 of the membrane 300. For example, as shown in FIG. 2, the coating material 400 may be coated on the upper surface and the lower surface of the membrane 300. Accordingly, the pores 310 of the membrane 300 may be covered with the coating material 400, and thus cells cultured in the first culture channel 140 and/or the second culture channel 240 may be prevented from unintentionally moving through the pores 310.

In an embodiment, as the coating material 400, materials in various phases may be used. For example, as the coating material 400, a material in a sol state in which solid particles are dispersed in a liquid and a material in a gel state, which has a certain shape in a solid or semi-solid state by gelation of the sol, may all be used. In particular, the coating material 400 may be a hydrogel and a material in a sol state in a pre-hydrogel stage.

A method of gelation is not particularly limited, and various methods, such as a method of heating or cooling a sol-state material at an appropriate temperature, a method of adding a linker, a method of performing ultraviolet (UV) irradiation, a method of mixing calcium chloride, or the like, may be used according to a type of material.

For example, the coating material 400 may be a hydrogel derived from an extracellular matrix (hereinafter also referred to as an ‘ECM hydrogel’), a naturally derived hydrogel, a synthetic hydrogel, a composite of one or more natural hydrogels and synthetic hydrogels, or a sol-state material in the pre-hydrogel stage.

For example, a composition of the coating material 400 may include and use an ECM-based biomaterial, such as collagen, gelatin, laminin, elastin, proteoglycan, glycosaminoglycan, albumin, vitronectin, heparin, heparan sulfate, hyaluronic acid, chondroitin sulfate, dermatan sulfate, keratan sulfate, tenascin, fibronectin, fibrinogen, or fibrin, and one or more of subgroups thereof. However, the disclosure is not limited thereto. A coating material may include one or more polysaccharide-based substances, such as starch, alginic acid, agar, agarose, carrageenan, cellulose, carboxymethylcellulose, chitosan, chitin, dextran, dextran salts, fluoran, pectin, keratin, silk fibroin, silk sericin, silica, lignin, guar gum, xanthan gum, gum arabic, locust bean gum, natto gum, galactomannan, glucomannan, gellan gum, or other naturally derived polymers, or a hydrogel based on a synthetic polymer including a copolymer with a hydrophilic group, such as polyacrylic acid (PAA), polyacrylonitrile (PAN), polyacrylamide (PAM), polyamidoamine (PAMAM), polymethacrylic acid (PMAA), polymethyl methacrylate (PMMA), polybutylene succinate (PBS), polycarbonate (PC), polycaprolactone (PCL), polydopamine (PDA), polydiethylaminoethyl methacrylate (PDEAEMA), polydiallyldimethylammonium chloride (PDADMAC), polyethylene (PE), polyethylene terephthalate (PET), polyethylene glycol (PEG), polyethylene oxide (PEO), polyethyleneimine (PEI), polyethylene glycol methacrylate (PEGMA), polyethylene glycol dimethacrylate (PEGDMA), polyglycerol (PG), polyglycolic acid (PGA), polyhydroxyalkanoate (PHA), polyhydroxybutyrate (PHB), Polyhydroxyethyl methacrylate (PHEMA), polylactic acid (PLA), polylactic coglycolic acid (PLGA), poly(N-isopropylacrylamide (PNIPA), polyphosphazene (PPHO), polyphosphoester (PPE), polypropylene oxide (PPO), polyurethane (PU), polyvinyl alcohol (PVA), polyvinylpyrrolidone (PVP), polysiloxane, poloxamer, poloxamine, or the like, or a sol-state material in the pre-hydrogel stage, and a mixture of one or more of the above materials may be used. In addition to the above compositions, a coating material composition may include all types of synthetic and natural compound-based hydrogels, which may condense after gelation or condense in a non-gel state due to external stimuli and factors, such as temperature and external force, and a sol-state material in the pre-hydrogel stage.

Also, the coating material composition may be a mixture of one or more of the above compositions.

In addition, the coating material 400 may include various types of hydrogels with excellent biocompatibility and sol-state materials in the pre-hydrogel stage. In an embodiment, the coating material 400 may form a multi-layered structure on the membrane 300.

For example, a multi-layered structure may be formed by coating the coating material 400 on the membrane 300 and then drying the same, coating the coating material 400 on the membrane 300 and then gelling and drying the same, or performing both of the two methods.

For example, the coating material 400 may be primarily coated on the membrane 300 and then be gelled and condensed, and the same or different coating material 400 may be coated multiple times on the condensed coating material 400 and then be gelled and condensed, thereby forming a multi-layered structure.

In another embodiment, a coating material 400 that does not gel may be used. For example, the coating material 400 is primarily coated on the membrane 300 and then is gelled and condensed. Thereafter, another coating material 400 that does not gel may be coated and condensed on the condensed coating material 400, and the above process may be repeated multiple times. In another embodiment, the coating material 400 may be formed in a multi-layered structure by repeating the coating and condensing of a coating material that does not gel by multiple times. That is, the membrane 300 or the microfluidic chip 10 including the same according to an embodiment may have a single-layered or multi-layered structure including the coating material 400 that gels and/or the coating material 400 that does not gel.

In an embodiment, a functional membrane may be formed by forming the coating material 400 on the membrane 300.

Accordingly, a functional membrane according to an embodiment may include the membrane 300 having one or more pores and the coating material 400 arranged on at least one surface of the membrane 300 to cover the pores. Also, the functional membrane according to an embodiment may form a multi-layered structure on the membrane 300.

A concentration of the coating material 400 may be 0.1 mg/ml to 10 mg/mL. More preferably, a concentration of the coating material 400 may be 0.125 mg/mL to 2 mg/mL. By making the concentration of the coating material 400 in the above range, a thickness of the coating material 400, which is dried, that is, condensed, may be maintained at 500 nm or less. Accordingly, a thin basement membrane of a human body may be better simulated. Also, molecular and cellular interactions between cells cultured on upper and lower portions of the membrane 300 may be promoted by adjusting the concentration of the coating material 400 to adjust a fibrous structure or density of the condensed coating material 400 to adjust material permeability, or the like.

Through the above configuration, the functional membrane and the microfluidic chip 10 including the same according to an embodiment may have pores each having a sufficient diameter formed in the membrane 300, and thus cells may be easily observed through a microscope without scattering of light. Also, in the functional membrane and the microfluidic chip 10 including the same according to an embodiment, as the pores 310 of the membrane 300 are covered with the coating material 400, cells cultured in different culture channels do not unintentionally move through the pores at the beginning of culture, and thus tissues including different cells in an attached state may be easily cultured. In addition, in the functional membrane and the microfluidic chip 10 including the same according to an embodiment, instead of forming a membrane by the coating material 400 itself, as the coating material 400 is coated on the membrane 300 having a hardness of a certain level or more, the functional membrane has excellent mechanical strength and is easy to maintain its shape as compared to a case where a membrane includes only a gel or sol-state material.

In an embodiment, cells cultured in the microfluidic chip 10 according to an embodiment may be cultured dividedly in each of the first culture channel 140 and the second culture channel 240 at the beginning of culture. Thereafter, the culture is performed, and according to stimulation involved in movement of cells, including a concentration gradient of cell attractants, immune and inflammatory reaction, or the like, the cells may decompose the coating material 400 or move between the gaps of fibers of the coating material 400, and thus cell movement may occur while the cells move to neighboring culture channels through the pores 310. That is, the microfluidic chip 10 according to an embodiment may culture cells separately from each other at the beginning of culture, and then as the culture is performed, a state in which the cells move and/or are coupled to each other by passing through a membrane may be observed.

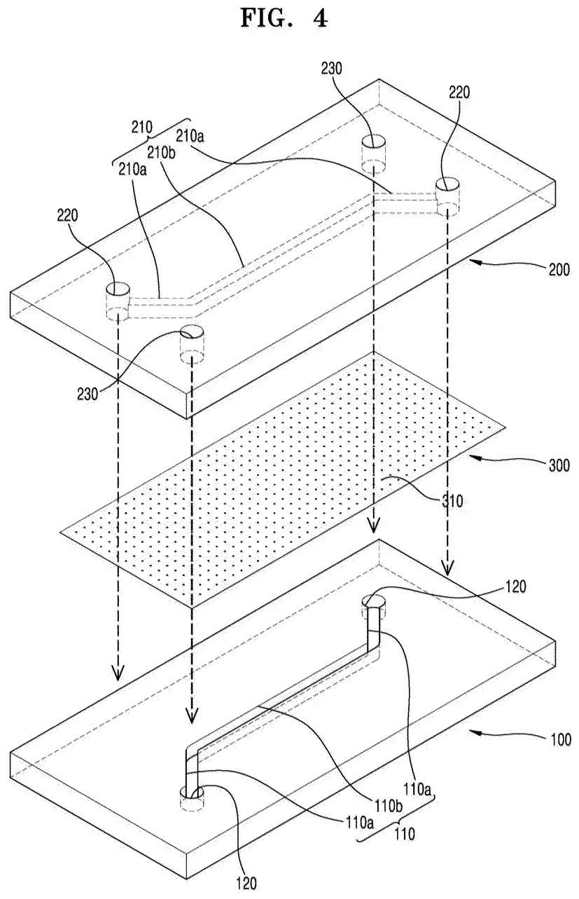

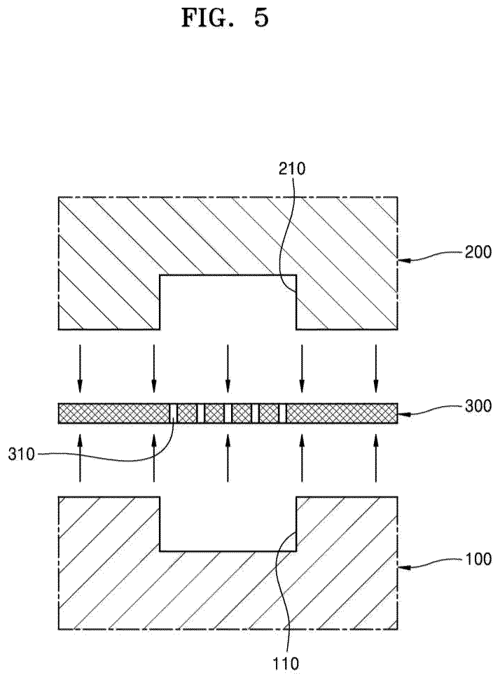

FIGS. 4 to 14 show a method of manufacturing the microfluidic chip 10 according to another embodiment.

The method of manufacturing the microfluidic chip 10 according to an embodiment may include preparing one or more substrates with patterned culture grooves, preparing the membrane 300 having one or more pores, arranging the membrane 300 between substrates, and injecting the coating material 400 into the culture grooves to apply the coating material 400 on at least one surface of the membrane 300 and drying the coated coating material 400 to arrange, on at least one surface of the membrane 300, the coating material 400 covering the pores.

First, a substrate with a patterned culture groove is prepared. For example, as shown in FIG. 4, the first substrate 100 in which the first culture groove 110 is patterned and the second substrate 200 in which the second culture groove 210 is patterned may be prepared. In an embodiment, the first substrate 100 and the second substrate 200 may each be a PMMA substrate.

Next, the membrane 300 is prepared. For example, as shown in FIG. 4, the membrane 300 having the pores 310 may be prepared. In an embodiment, the membrane 300 may be a polyethylene terephthalate (PETE) membrane.

In an embodiment, performing a separate surface treatment on the membrane 300 may be further included. For example, the membrane 300 may be treated with air plasma (e.g., 80 W, 5 kHz) for 1 minute and then put into an additive (e.g., 5% GLYMO (94% ethanol solvent)) heated to 100° C. to perform surface treatment for 30 minutes.

Next, the membrane 300 may be arranged between the prepared substrates. For example, as shown in FIGS. 4 and 5, the first substrate 100 and the second substrate 200 may be arranged to face each other, and then the membrane 300 may be arranged therebetween. Here, the membrane 300 may be in a state in which surface treatment is performed thereon.

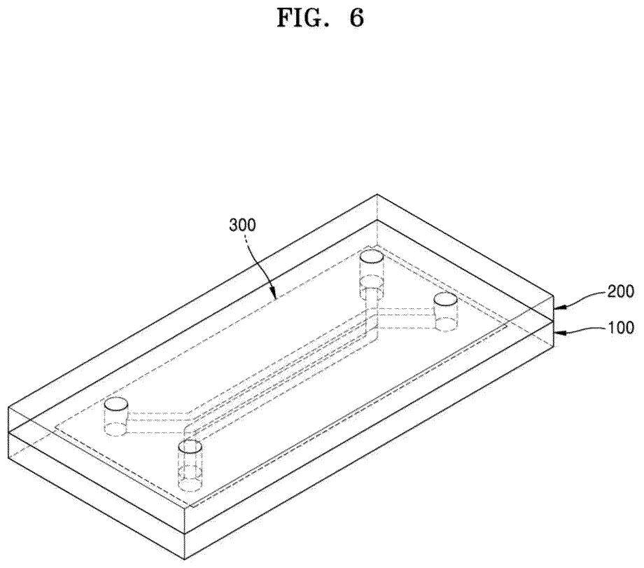

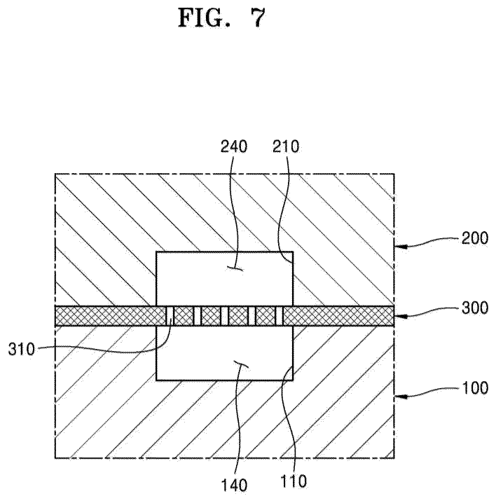

Also, the first substrate 100, the second substrate 200, and the membrane 300 may be fixed to each other by heating and adhering for a certain period of time (refer to FIG. 6). For example, the first substrate 100, the membrane 300, and the second substrate 200 may be sequentially fixed and then adhered to each other by coating heat of 100° C. for 4 minutes, thereby completely fixing the first substrate 100, the membrane 300, and the second substrate 200. Accordingly, the first culture channel 140 and the second culture channel 240 may be divided between the first substrate 100, the second substrate 200, and the membrane 300 (refer to FIG. 7).

In an embodiment, cleaning the first substrate 100, the second substrate 200, and the membrane 300 may be further included. For example, a cleaning solution (e.g., 70% ethanol and phosphate-buffered saline (PBS)) may be injected through injection holes formed in the first substrate 100 and the second substrate 200 to process various chemical solutions remaining on the first substrate 100, the second substrate 200, and the membrane 300.

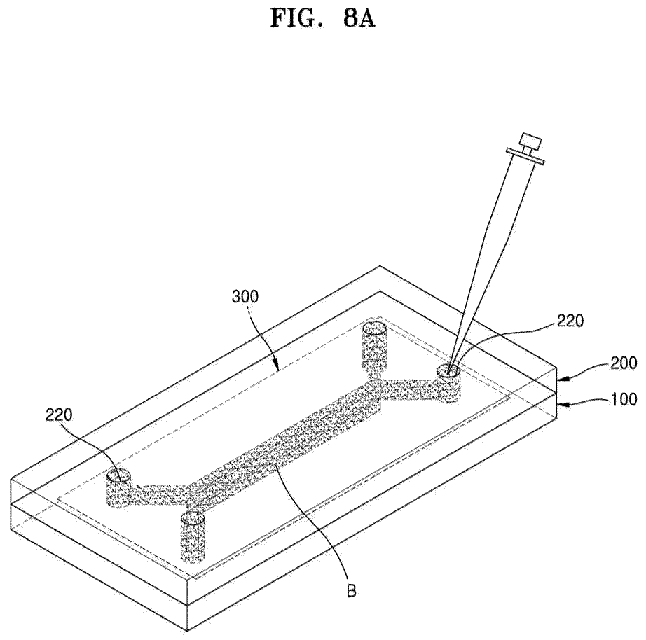

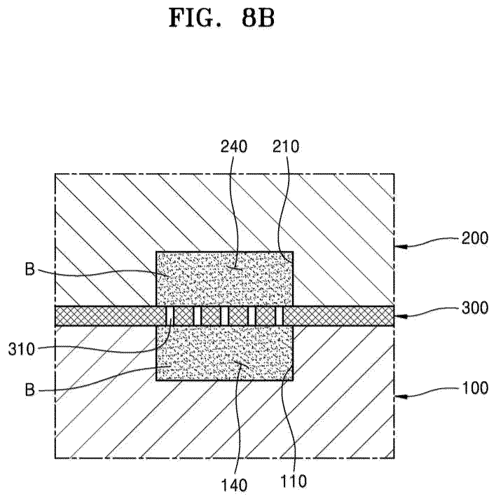

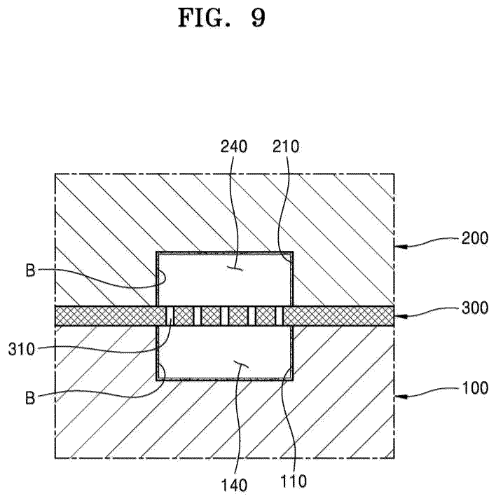

In an embodiment, performing blocking treatment on an inner portion of the culture groove may be further included. For example, as shown in FIG. 8A, respective connection holes 230 of the second substrate 200 may be connected to respective first injection ports 120 of the first substrate 100, and accordingly, a blocking solution (e.g., BSA) may be injected into the first culture groove 110 through the connection hole 230. Alternatively, a blocking solution may be injected into the second culture groove 210 through the second injection portion 220 of the second substrate 200. Thereafter, in a state in which the blocking solution is injected into the inner portions of the first culture groove 110 and the second culture groove 210, the inner portions of the first culture groove 110 and the second culture groove 210 may be subjected to blocking treatment by reacting in an incubator for a certain period of time (e.g., 1 hour) or reacting at room temperature (refer to FIG. 8). Also, the blocking solution may all be sucked through a suction device (e.g., a vacuum aspirator) to empty the inner portions of the first culture groove 110 and the second culture groove 210 (refer to FIG. 9).

Accordingly, the inner portions of the first culture groove 110 and the second culture groove 210 may be subjected to blocking treatment, and cells may be aligned with the coating material 400 coated on the membrane 300 to be cultured without being cultured on inner surfaces of the first culture groove 110 and the second culture groove 210 during a cell culture process.

In an embodiment, before the f arranging of the coating material 400, performing pretreatment on all or part of the membrane 300 to have a higher surface energy than the surface energy of the first substrate 100 and the second substrate 200 (e.g., the first culture groove 110 and the second culture groove 210) may be further included. For example, the membrane 300 may become more hydrophilic than the first substrate 100 and the second substrate 200 after the pretreatment. Alternatively, the membrane 300 may include a material that has greater hydrophilicity than materials of the first substrate 100 and the second substrate 200.

Accordingly, after the coating material 400 is arranged on the membrane 300, the coating material 400 may not move to the first culture groove 110 of the first substrate 100 and/or the second culture groove 210 of the second substrate 200 and may be led to the membrane 300 having higher hydrophilicity and surface energy.

A method of performing pretreatment on the first substrate 100 and the first culture groove 110 and/or the second substrate 200 and the second culture groove 210 is not particularly limited. For example, by performing plasma treatment on the microfluidic chip 10 including the first substrate 100, the second substrate 200, and the membrane 300, a surface energy of the membrane 300 therein may be greater than the surface energy of the first substrate 100 and the first culture groove 110 and/or the second substrate 200 and the second culture groove 210. Here, a gas used in plasma treatment may be a gas such as oxygen (O₂), argon (Ar), nitrogen (N₂), or the like, synthetic air (e.g., e.g. 80% nitrogen (N₂)+20% oxygen (O₂)), or a mixed gas of argon and hydrogen (e.g., 90% argon (Ar)+10% hydrogen (H₂)), moist oxygen (O_{2d}H₂O), or the like. However, an optimal gas mixture for plasma treatment is not limited to the above gases because the optimal gas mixture varies depending on temperature and external conditions, and may include all gases and mixed gases that may improve hydrophilicity and adhesiveness of the surface of a polymer material when activated by plasma. In addition, a material for which surface treatment is desired is firstly plasma treated with organosiloxane gases (but not limited thereto), such as TMDSO, HMDSO, TEOS, and HMCTSO, and then is additionally plasma treated with gases such as oxygen, argon, nitrogen, or the like to further improve the hydrophilicity and adhesiveness of the material surface.

In an embodiment, the performing of pretreatment may include performing blocking treatment on the first substrate 100 and/or the second substrate 200. In more particular, bottom surfaces and side surfaces of the first culture groove 110 and the second culture groove 210 may be treated with a blocking solution (e.g., BSA), and thus the coating material 400 may not be coated to the inner surfaces of the first culture groove 110 and the second culture groove 210. Accordingly, cells may not be arranged on the inner surfaces of the first culture groove 110 and the second culture groove 210, which are not coated with the coating material 400.

In addition, various methods of leading the coating material 400 to the membrane 300 by using a surface energy difference may be used. That is, various methods may be used to perform pretreatment on at least a portion of the membrane 300 to have a greater surface energy than the surface energy of the first substrate 100 and the second substrate 200.

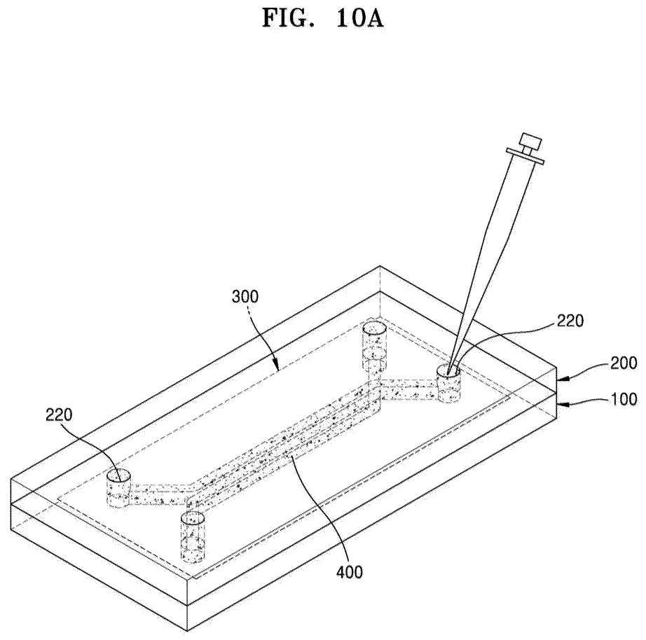

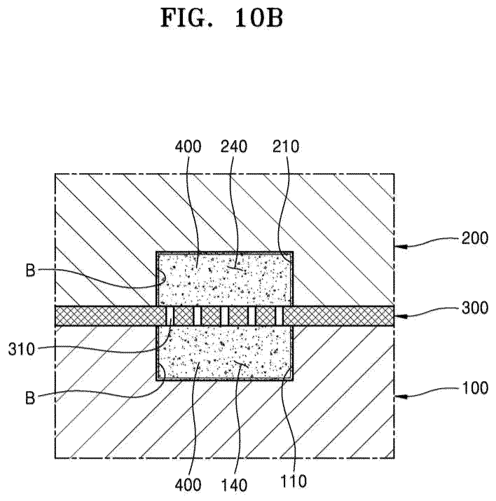

Next, the coating material 400 may be arranged on at least one surface of the membrane 300 to cover the pores 310. For example, as shown in FIG. 10, respective connection holes 230 of the second substrate 200 may be connected to respective first injection ports 120 of the first substrate 100, and accordingly, the coating material 400 may be injected into the first culture groove 110 through the connection hole 230. Alternatively, the coating material 400 may be injected into the second culture groove 210 through the second injection portion 220 of the second substrate 200 (refer to FIG. 10).

Here, the first culture groove 110 and the second culture groove 210 may each be in a state in which blocking treatment is performed thereon. Accordingly, the coating material 400 may be coated only on the membrane 300 without being coated on the inner surfaces of the first culture groove 110 and the second culture groove 210.

In an embodiment, the arranging of the coating material 400 may further include gelling the coated coating material 400.

In an embodiment, the arranging of the coating material 400 may further include injecting the coating material 400 into a culture groove and then reacting and gelling the injected coating material 400 for a certain period of time, and drying the gelled coating material 400 for a certain period of time.

In more particular, when the coating material 400 is injected into the first culture groove 110 and the second culture groove 210, the coating material 400 is reacted in an incubator for a certain period of time (e.g., 1 hour) to gel the coating material 400 in the inner portions of the first culture groove 110 and the second culture groove 210 (refer FIG. 10).

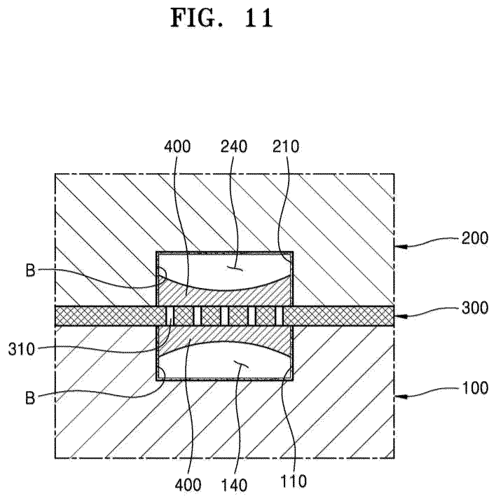

Next, the gelled coating material 400 is dried for a certain period of time (e.g., one day or more) to apply the coating material 400 on the membrane 300. In an embodiment, as the coating material 400 is dried to dry the moisture in the coating material 400, the coating material 400 injected into the first culture groove 110 and the second culture groove 210 may be condensed. As described above, in the pretreatment process, the membrane 300 may be a material that may become more hydrophilic than the first culture groove 110 and the second culture groove 210 and may be subjected to pretreatment (e.g., blocking or plasma treatment) to have a higher surface energy than the surface energy of the first culture groove 110 and the second culture groove 210, and thus the coating material 400 may be condensed on the membrane 300 according to drying (refer to FIG. 11).

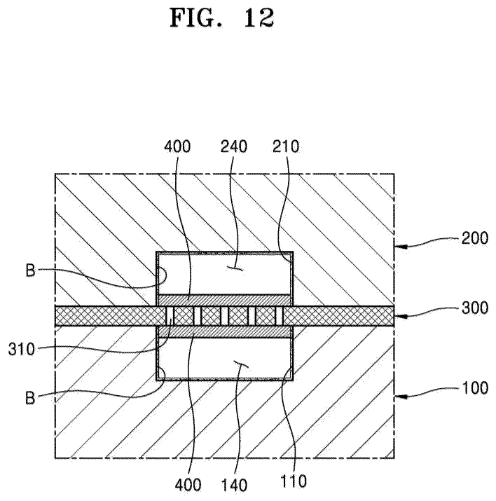

Also, when drying is completed, the coating material 400 may form a condensed layer on the membrane 300 (refer FIG. 12). Accordingly, the pores 310 formed in the membrane 300 may be covered with the coating material 400, and thus cells cultured in any one culture channel may be prevented from unintentionally moving to another culture channel at the beginning of culture.

In an embodiment, the arranging o the coating material 400 may further include checking whether bubbles have been generated inside a culture groove after injecting the coating material 400 into the culture groove and removing the generated bubbles.

FIGS. 10 to 12 show that the coating material 400 is injected into both of the first culture groove 110 and the second culture groove 210 and the coating material 400 is condensed and coated on both surfaces of the membrane 300, but the disclosure is not limited thereto. For example, the coating material 400 may be injected only into ay one culture groove, and thus the coating material 400 may be condensed and coated only on one surface of the membrane 300.

In an embodiment, coating or arranging and condensing the coating material 400 may be repeated multiple times, and thus the coating material 400 may form a multi-layered structure.

For example, the multi-layered structure may be formed by coating the coating material 400 on the membrane 300 and then drying the same, coating the coating material 400 on the membrane 300 and then gelling and drying the same, or performing both of the two methods.

For example, the coating material 400 may be primarily coated on the membrane 300 and then be gelled and condensed, and the same or different coating material 400 may be coated multiple times on the condensed coating material 400 and then be gelled and condensed, thereby forming the multi-layered structure.

In another embodiment, a coating material 400 that does not gel may be used. For example, the coating material 400 is primarily coated on the membrane 300 and then is gelled and condensed. Thereafter, another coating material 400 that does not gel may be coated and condensed on the condensed coating material 400, and the above process may be repeated multiple times.

In another embodiment, the coating material 400 may be formed in a multi-layered structure by repeating coating and condensing a coating material that does not gel by multiple times. That is, the membrane 300 or the microfluidic chip 10 including the same according to an embodiment may have a single-layered or multi-layered structure including the coating material 400 that gels and/or the coating material 400 that does not gel.

In an embodiment, arranging the coating material 400 may include adjusting the concentration of the coating material 400 to adjust at least one of the thickness, density, pore size, and permeability of the dried coating material 400. For example, as the concentration of the coating material 400 decreases, the thickness of the dried, that is, condensed, coating material 400 may decrease, the density thereof may decrease, and the pore size and permeability thereof may increase. Here, permeability may mean permeability of materials and cells. In an embodiment, arranging the coating material 400 may include arranging the coating material 400 at a concentration of 0.125 mg/ml to 2 mg/ml. Accordingly, the thickness of the condensed coating material 400 may be 500 nm or less. Here, the coating material 400 may be a collagen type I.

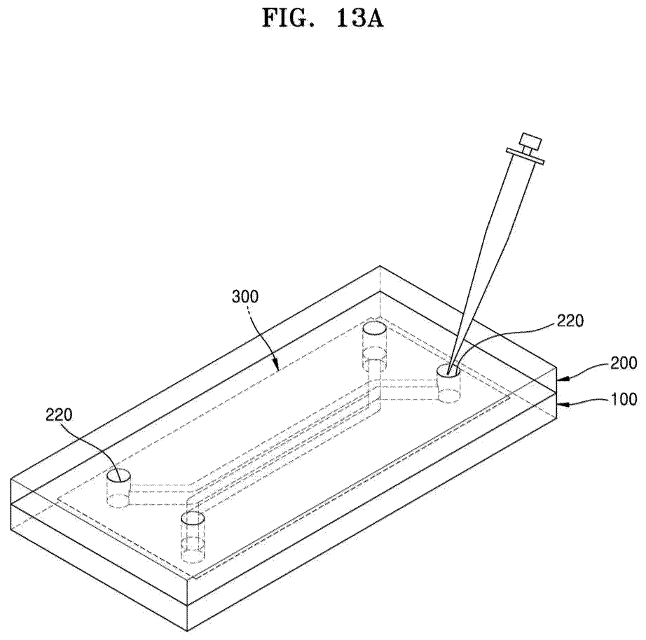

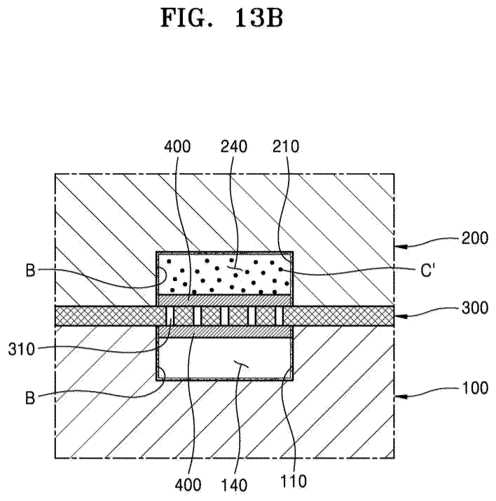

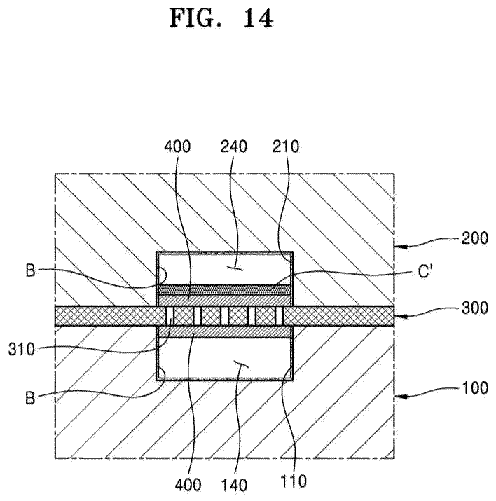

Next, cell culture may be performed by using the microfluidic chip 10. For example, as shown in FIG. 13A, cells may be injected into the second culture groove 210 through the second injection portion 220. Also, the cells may be cultured in the second culture channel 240. Also, as described above, as the membrane 300 is covered with the coating material 400, cells injected into the second culture groove 210 may not intentionally move to the first culture channel 140 through the pores 310 (refer to FIGS. 13B and 14).

In an embodiment, a functional membrane may be manufactured by coating the coating material 400 on the membrane 300 without using a substrate. That is, the functional membrane may be manufactured by using only the membrane 300 and the coating material 400.

For example, a method of manufacturing the functional membrane may include preparing the membrane 300 having one or more pores and coating the coating material 400 on at least one surface of the membrane 300 and drying the coated coating material 400 to arrange the coating material 400, which covers the pores, on at least one surface of the membrane 300.

In an embodiment, the arranging of the coating material 400 may further include gelling the coated coating material 400.

In an embodiment, the arranging of the coating material 400 may form a multi-layered structure by arranging the coating material 400 multiple times.

In an embodiment, the arranging of the coating material 400 may further include reacting the coating material 400 for a certain period of time to gel the coating material 400.

In an embodiment, the arranging of the coating material 400 may form the coating material 400 in a multi-layered structure by repeating the gelling and condensing of the coating material 400 on the membrane 300 by multiple times.

In an embodiment, in the arranging of the coating material 400, the coating material 400 may be coated on at least one surface of the membrane 300 to be gelled and condensed, and then the coating material 400 that does not gel may be coated and condensed on the condensed coating material 400, and the above operations may be repeated multiple times. In another embodiment, the coating material 400 may be formed in a multi-layered structure by repeating coating and condensing a coating material that does not gel by multiple times.

In more particular, in the arranging of the coating material 400, coating and gelling the coating material 400 on at least one surface of the membrane 300 and then condensing the gelled coating material 400, coating the coating material 400 of a non-gelling type or a gelling type on the condensed coating material 400 and then condensing or gelling to condense the coated coating material 400 may be repeated. That is, a multi-layered structure may be formed by repeating a process of gelling and condensing the coating material 400 and then coating and condensing the coating material 400 that does not gel or coating to gel and condense the coating material 400 that gels thereon.

Alternatively, in the arranging of the coating material 400, coating the coating material 400 that does not gel on at least one surface of the membrane 300 and then condensing the coated coating material 400, coating the coating material 400 of a non-gelling type or a gelling type on the condensed coating material 400 and then condensing or gelling to condense the coated coating material 400 may be repeated. That is, the multi-layered structure may be formed by repeating a process of condensing the coating material 400 that does not gel and then again coating and condensing the coating material 400 that does not gel or coating and gelling the coating material 400 that gels thereon.

In an embodiment, the arranging of the coating material 400 may include adjusting the concentration of the coating material 400 to adjust at least one of the thickness, density, pore size, and permeability of the dried coating material 400. For example, as the concentration of the coating material 400 decreases, the thickness of the dried, that is, condensed, coating material 400 may decrease, the density thereof may decrease, and the pore size and permeability thereof may increase. Here, permeability may mean permeability of materials and cells. In an embodiment, the arranging of the coating material 400 may include arranging the coating material 400 at a concentration of 0.125 mg/ml to 2 mg/ml. Accordingly, the thickness of the condensed coating material 400 may be 500 nm or less. Here, the coating material 400 may be a collagen type I.

In an embodiment, the coating material 400 may be a material that is in a gel state or a sol-state before being gelled.

Accordingly, the method of manufacturing the functional membrane according to an embodiment may form a single-layered or multi-layered structure including the coating material 400 that gels and/or the coating material 400 that does not gel on the membrane 300.

In an embodiment, in the method of manufacturing the microfluidic chip 10, after the membrane 300 is manufactured according to the method of manufacturing the functional membrane described above, the membrane 300 may be arranged between substrates, and then the above operations may be performed. That is, when the arranging of the membrane 300 after the substrates are completed to arrange the coating material 400 in the microfluidic chip 10 is performed in the method of manufacturing the microfluidic chip 10 described above, unlike the above case, arranging the coating material 400 on the membrane 300 may be performed separately outside the microfluidic chip 10. The method of manufacturing the microfluidic chip 10 according to the above embodiment may include manufacturing the membrane 300, preparing substrates with patterned grooves, and arranging the membrane 300 between the substrates. Also, the arranging of the coating material 400 may be performed in the manufacturing of the membrane 300. The method of manufacturing the microfluidic chip 10 according to the embodiment is similar to the method of manufacturing the microfluidic chip 10 described above, except for an order of each operation and a point that the coating material 400 is separately arranged on the membrane 300 outside the microfluidic chip 10, and detailed descriptions thereof are omitted.

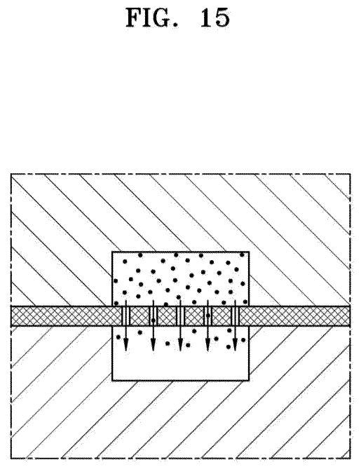

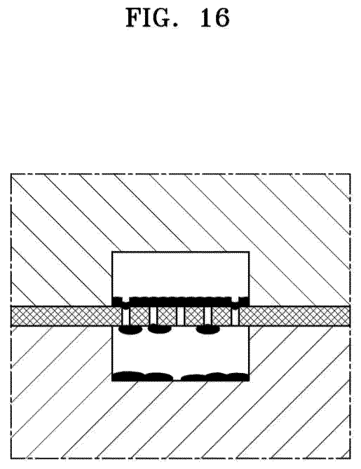

FIGS. 15 and 16 show a microfluidic chip according to a comparative example.

As shown in FIGS. 15 and 16, the microfluidic chip according to the comparative example have pores formed on a membrane, but no member covering the pores is arranged. Accordingly, some of cells injected into an upper channel are cultured in the upper channel but some other cells unintentionally move to a lower channel through the pores. Also, as time passes, cells are cultured on both surfaces of the membrane as well as a lower surface of the lower channel.

That is, in the case of the microfluidic chip according to the comparative example, unlike the microfluidic chip 10 according to the embodiment, because cells move to another culture channel from the beginning of cell culture, different cells may not be cultured in different culture channels. Also, tissues formed by combining cells cultured in different cell culture regions may not be properly implemented.

In the microfluidic chip 10 according to an embodiment and a method of manufacturing the microfluidic chip 10, an unit capable of efficiently simulating an actual biological environment regardless of a pore size of a membrane while having excellent optical characteristics may be provided by providing the membrane with pores of sufficient size.

In the microfluidic chip 10 according to an embodiment and a method of manufacturing the microfluidic chip 10, cells may be clearly observed without light scattering on the membrane 300 by providing large pores of 3 □ or more (or 10 □ or more), as compared with a microfluidic chip in the related art, which provides small pores of 3 □ or less.

In the microfluidic chip 10 according to an embodiment and a method of manufacturing the microfluidic chip 10, as the membrane 300 is covered with the coating material 400 while having pores of large size, cells in different culture channels may be prevented from unintentionally moving to other culture channels at the beginning of cell culture. Accordingly, different cells may be cultured in different culture regions.

In the microfluidic chip 10 according to an embodiment and a method of manufacturing the microfluidic chip 10, after cell culture is sufficiently performed, cells may naturally decompose the coating material 400 with enzymes through stimuli involved in cell movement, including concentration gradients of cell attractants, inflammation, immune responses, or the like, or movement between cells or connections between cells through pores, including movement between gaps of fibers of the coating material 400, may be induced.

### Example 1

To compare characteristics such as thickness and structure of an ECM fiber membrane according to a concentration of an ECM hydrogel condensed and coated on a membrane within a microfluidic chip, according to an embodiment, a microfluidic chip including a membrane was manufactured, and then an ECM (e.g., collagen type I) hydrogel at concentrations of 2 mg/mL, 1 mg/mL, 0.125 mg/mL, and 0.06 mg/mL were respectively injected into the membrane as a coating material. Thereafter, the hydrogel was dried and condensed at room temperature in a sterile space. Next, the microfluidic chip on which the ECM was condensed and coated was disassembled, the membrane was taken out, and then an image of a surface of the membrane was captured by a scanning electron microscope (SEM).

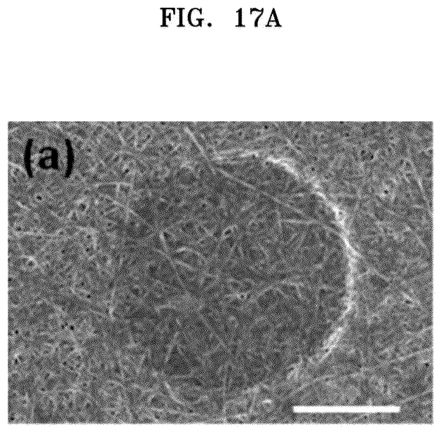

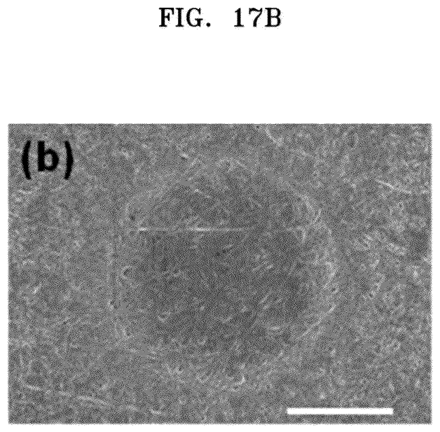

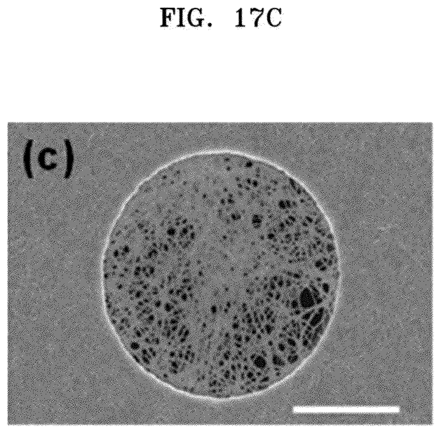

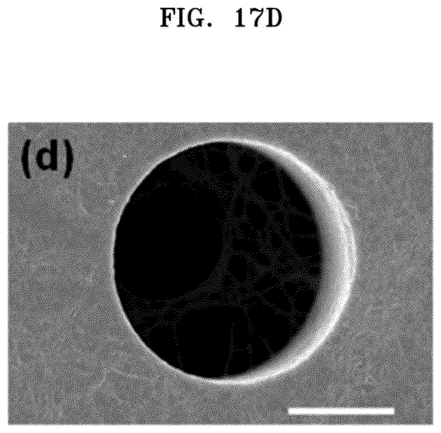

FIGS. 17A to 17D show captured images of pores of membranes in an order that coating materials at concentrations of 2 mg/mL, 1 mg/mL, 0.125 mg/mL, and 0.06 mg/mL were used. As shown in FIGS. 17A to 17D, it may be seen that a difference exists in a nanofiber structure formed on a surface of a membrane according to a concentration of the coating material. In more particular, it may be seen that as the concentration of the coating material increases, a thickness and density of a fiber film formed increases, and as the concentration of the coating material decreases, gaps between a fibrous structure become wider, and larger pores are made to increase material and cell permeability.

In this way, in the disclosure, a concentration of the coating material may be adjusted to adjust a thickness and density of a fiber film formed on a membrane and adjust a porous structure and permeability of a surface of the fibrous film.

### Example 2

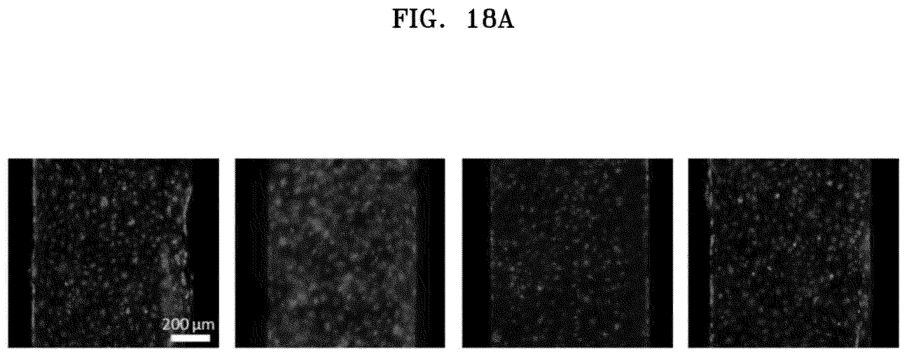

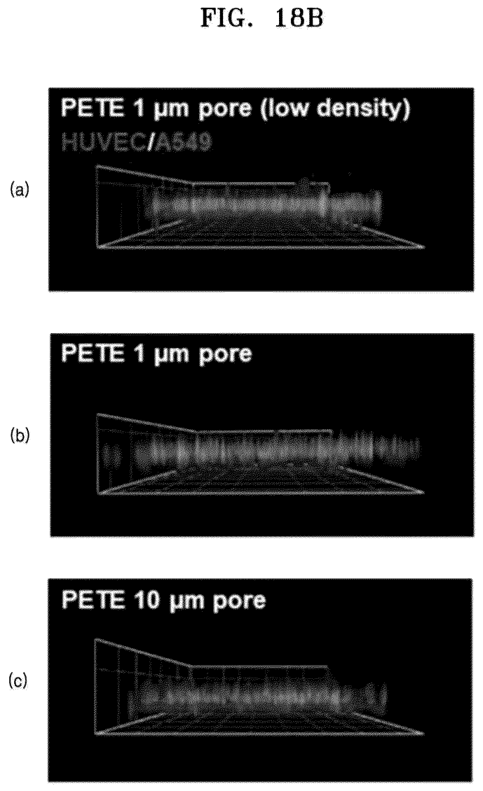

To check optical characteristics, such as an autofluorescence expression level, light transmittance, scattering, or the like, according to a pore size and density of a porous membrane in a microfluidic chip having condensed and coated ECM, according to an embodiment, a microfluidic chip including a membrane having a relatively high pore density and a pore size of 1 μm, a microfluidic chip including a membrane having pore sizes of 5 □ to 10 □, and a microfluidic chip including a membrane including a membrane having a relatively low pore density and a pore size of 1 μm were manufactured. Thereafter, GFP-expressing human umbilical vein endothelial cells (GFP-HUVEC) were cultured on each microfluidic chip, and cell images were captured by a fluorescence microscope (refer to FIG. 18A). Also, after human lung cancer epithelial cells (A549) were co-cultured with the human umbilical vein endothelial cells, 3D images of the cells were captured by using a confocal microscope (refer to FIG. 18).

In particular, GFP-HUVEC and A549 were respectively cultured with an endothelial cell (EC) medium containing 10% FBS and 1% antibiotics and a Ham's F12 medium containing 10% FBS and 1% antibiotics in an incubator at 37° C. and 5% CO₂ conditions. The mediums were changed once every two to three days, and when the cells were about 80% full in a flask, subculture were performed by using 0.25% trypsin/ethylenediaminetetraacetic acid (EDTA) solution.

An inner portion of a channel of the microfluidic chip was washed with 1×PBS and rehydrated for 30 minutes. Thereafter, GFP-HUVEC (3×10⁶ cells/mL) were injected into an upper channel of the microfluidic chip by using a micropipette and cell attachment was performed during one hour, and then, in the incubator (37° C., 5% CO₂), the microfluidic chip was connected to a syringe pump, and the EC medium was flowed at a rate of 2 μL/min for 48 hours.

For A549 co-culture, after attaching GFP-HUVEC to the upper channel of the microfluidic chip through the above process, A549 (5×10⁶ cells/mL) were injected into a lower channel of the microfluidic chip and the chip was flipped to perform cell attachment for 1 hour. Thereafter, the microfluidic chip was connected to a syringe pump in an incubator, and each cell medium was flowed at a rate of 2 μL/min for 48 hours. After completion of incubation, A549 was fluorescently stained with 5 μM CellTracker™ Red in an incubator for 30 minutes.

FIG. 18A shows a 2D image of a GFP-HUVEC cell layer, and FIG. 18B shows a 3D image of GFP-HUVEC and A549 cell layers.

In general, as a pore density of a membrane decreases, light scattering according to pores may be reduced, thereby maintaining high optical transparency. In addition, as a pore size increases, material permeability of the membrane increases to lower the pore density, resulting in relatively high optical transparency.

As can be seen in FIGS. 18A and 18B, the disclosure maintains optical characteristics of a membrane according to a pore size and density of the membrane even after ECM was condensed and coated as a coating material.

In particular, as shown in FIG. 18A, it can be seen that, when a membrane with low pore density and small (1 □) pore size, which has low light scattering, or a membrane with large pore size (10 □) are used, compatibility with microscopes is excellent, for example, cell images are clearly captured even when ECM is condensed and coated. On the contrary, it can be seen that, in the case of a membrane having relatively high pore density and a pore size of 1 □ or a membrane having a pore size of 5 □, which does not have excellent optical characteristics, cell images may not be clearly captured even after condensing and coating.

Also, as shown in FIG. 18B, when a coating material is condensed, coated and used on a membrane having low pore density and a small pore size (1 □, (a) of FIG. 18B) or a membrane having a large pore size (10 □, (c) of FIG. 18B), different layers may be formed and separated, and the different layers may be clearly distinguished through a microscope. In particular, it can be seen that stable culture of cells is possible through a membrane having much higher material permeability and large pores, as compared to an existing membrane having a small pore size.

In this way, the disclosure may secure both high material permeability and excellent optical characteristics by condensing and coating ECM on a membrane having relatively large pores.

### Example 3

To check that a blood-brain barrier is well formed in a manufactured membrane and a microfluidic chip including the membrane and original characteristics of the blood-brain barrier are maintained, a cell layer and marker protein of the blood-brain barrier within the microfluidic chip were checked through immunofluorescence staining and fluorescence microscopy imaging, and a barrier value of the blood-brain barrier was checked through a permeability experiment by using lucifer yellow.

### 1) Induced Pluripotent Stem Cell Culture and Differentiation Into Vascular Endothelial Cells

The induced pluripotent stem cell (IMR90-4) were cultured in an incubator at 37° C. and 5% CO₂ conditions by using a TeSR™-E8™ medium. After a process of singularizing the cultured cells by using Accutase is performed, the cultured cells were divided into 1.7×10⁴ cells and cultured in one well of a 6-well plate, and a medium was changed every day for 3 days. From the fourth day, a cell medium was changed to an unconditioned medium (UM), and the medium was changed every day during 5 days in a hypoxic incubator (5% CO₂, 5% O₂). From the sixth day, to selectively culture only vascular endothelial cells, the cells were cultured with an EC medium for 2 days to complete differentiation into vascular endothelial cells.

The UM consists of 78.5% DMEM/F12, 20% Knockout™ Serum Replacement, 1% non-essential amino acids (100×), 0.5% GlutaMAX™ supplement, and 0.007% β-mercaptoethanol, and the EC medium consists of 1% human serum, 20 ng/ml basic fibroblast growth factor (bFGF), and 10 μM retinoic acid in a human endothelial cell serum free medium (HESFM).

### 2) Blood-Brain Barrier Microfluidic Chip Model

An additional ECM coating (e.g., 400 μg/mL collagen type IV and 100 μg/ml human fibronectin) solution for vascular endothelial cell culture to both channels of the microfluidic chip on which an ECM (e.g., collagen type I) is condensed and coated was added, and culture was performed overnight in an incubator at 37 ° C. and 5% CO₂ conditions.

Thereafter, after the coating solution was entirely absorbed and dried at room temperature in a sterile environment for 20 minutes, vascular endothelial cells (1.2×10⁷ cells/mL) were injected into the upper channel of the microfluidic chip by using a micropipette and were cultured in an incubator. After waiting for cell attachment for at least 6 hours, new medium was flowed toward an inlet of the microfluidic chip to avoid lack of nutrient and drying of of cells. After 24 hours of incubation, a vascular EC medium excluding bFGF and retinoic acid was used, and a medium was changed by flowing new medium toward the inlet of the microfluidic chip every 24 hours.

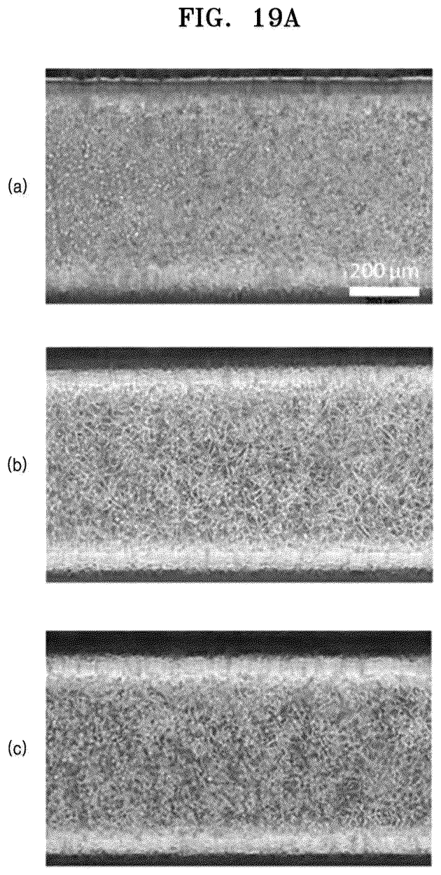

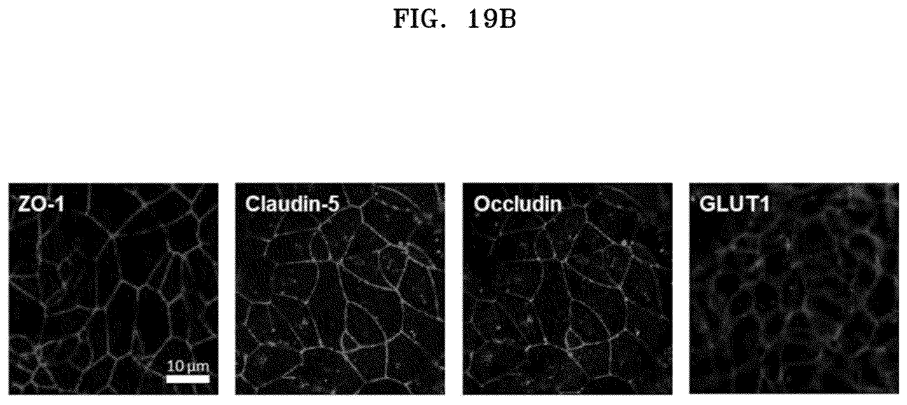

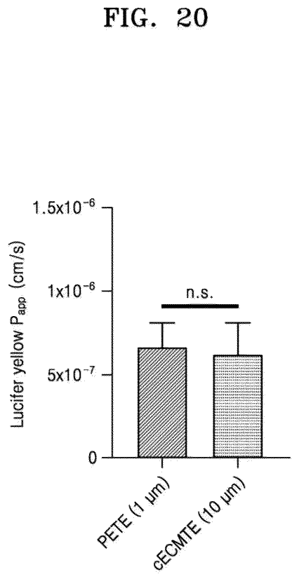

FIG. 19A shows images of a vascular endothelial cell layer (blood-brain barrier) over time after culture. In more particular, (a) to (c) of FIG. 19A respectively show 0 hours, 24 hours, and 48 hours after culture. FIG. 19B shows images of a vascular endothelial cell marker protein using immunofluorescence staining, and FIG. 20 shows a comparison of barrier permeability of the blood-brain barrier formed in a channel of the microfluidic chip.

As shown in FIG. 19A, when vascular endothelial cells at an optimized concentration (1.2×10⁷ cells/mL) were injected into the microfluidic chip, it can be seen that the vascular endothelial cells successfully formed intercellular junctions stably within 24 hours. On the contrary, vascular endothelial cells being cultured in an opposite side and a lower channel by passing through pores through a membrane having a pore size of 10 μm which is condensed and coated, was not observed.

Also, as shown in FIG. 19B, the formation of junction proteins, such as ZO-1, claudin-5, and occludin, at a boundary of vascular endothelial cells were checked through an immunofluorescence staining method, and the blood-brain barrier being properly formed was checked, for example, an expression of GLUT1, which is responsible for glucose transport across the blood-brain barrier, was also confirmed.

After manufacturing blood-brain barrier microfluidic chips through an existing porous membrane having a small pore size (polyester track-etched (PETE), 1 □) and a functional membrane of the disclosure (condensed-ECM-coated track-etched (cECMTE), 10 □), the barrier permeability of Lucifer yellow (457.3 g/mol) was analyzed on each chip to compare the formation of the blood-brain barrier (FIG. 20).

As shown in FIG. 20, it can be seen that, when a blood-brain barrier model optimized in small pores is used as a positive control to compare with a blood-brain barrier formed on a condensed-and-coated functional membrane, there was no significant difference in permeability between the two types of membranes.

Accordingly, it can be seen that, the blood-brain barrier formed through the functional membrane according to the disclosure and the microfluidic chip including the functional membrane may provide the same level of physical barrier as a blood-brain barrier model formed from an existing membrane of 1 □.

### Example 4

To check whether vascular endothelial cells and glial cells, which form a blood-brain barrier, interact well with each other in the functional membrane of the disclosure and the microfluidic chip including the functional membrane, a physical interaction between vascular endothelial cells and glial cells within the microfluidic chip was checked through immunofluorescence staining and confocal microscopy imaging, and an mRNA expression level of blood-brain barrier function genes extracted from the blood-brain barrier microfluidic chip of the disclosure was compared with an mRNA expression level of an existing blood-brain barrier microfluidic chip model.

In particular, a blood-brain barrier microfluidic chip was manufactured by co-culturing glial cells and vascular endothelial cells, before injecting the vascular endothelial cells, the glial cells (1×10⁶ cells/mL) were injected into a lower channel of the microfluidic chip by using a micropipette, and after cell attachment in an incubator for 2 hours, the vascular endothelial cells were injected as described in Example 3. Thereafter, a medium was changed by flowing the medium for each cell toward inlets of upper and lower channels of the microfluidic chip every 24 hours. After culture, the glial cells were fluorescently stained green by using a glial fibrillary acidic protein (GFAP) as a representative glial cell marker, and the vascular endothelial cells were fluorescently stained red by using ZO-1.

The glial cells were primary cells, and only cells below passage 4 were used in experiments, and cells were cultured in an incubator at 37° C. and 5% CO₂ conditions by using an astrocyte medium (AM) containing 2% FBS, 1% antibiotics, and 1×Astrocyte growth supplement (AGS). The mediums were changed once every two to three days, and when the cells were about 80% full in a flask, subculture were performed by using 0.25% trypsin/EDTA solution.

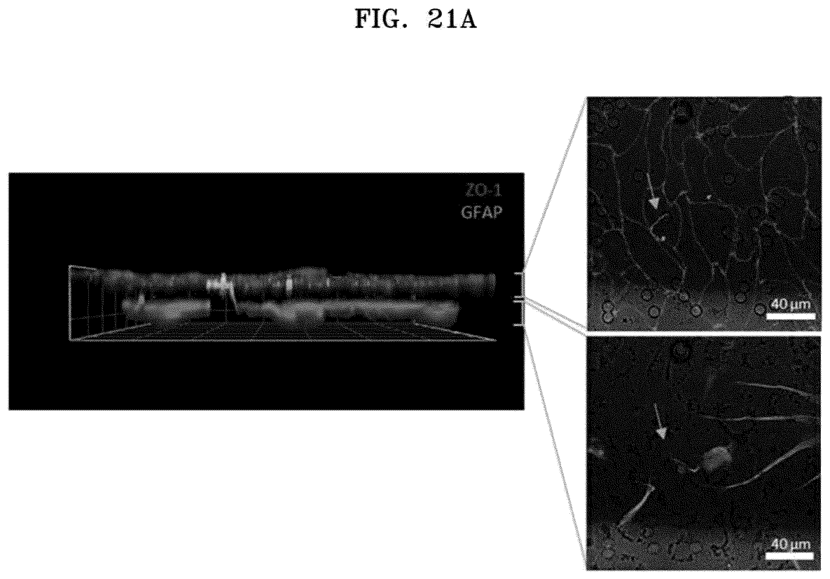

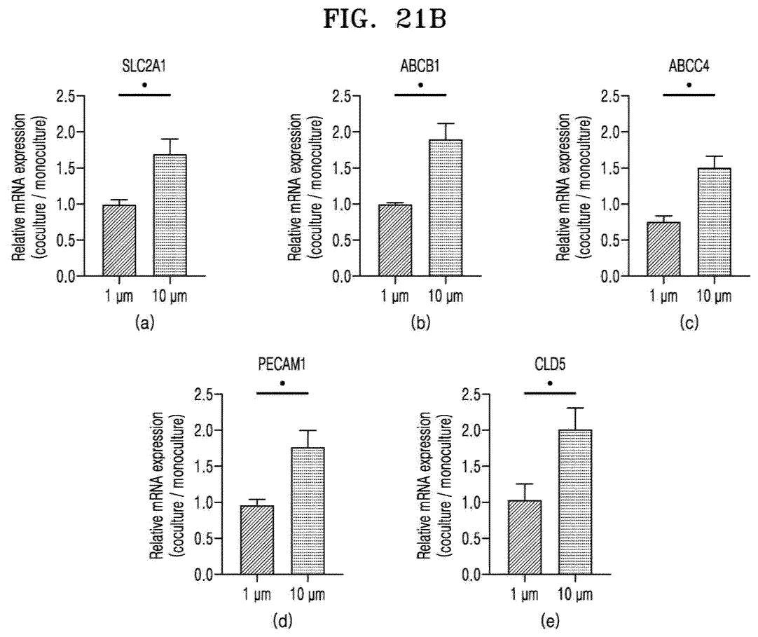

FIG. 21A shows a 3D image of a cell layer of vascular endothelial cells and glial cells, and FIG. 21B shows comparison of mRNA expression of blood-brain barrier function genes between microfluidic chip models.

A commonly used blood-brain barrier model manufactured through a membrane having small pore size (0.4 □ to 2 □) and low pore density allows only chemical interactions between vascular endothelial cells and glial cells.

As shown in FIG. 21A, in the case of a microfluidic chip including condensed-and-coated pores of 10 μm according to the disclosure, when an inner portion of the microfluidic chip is observed through a confocal microscope, it can be known that, the microfluidic chip structurally mimics better a blood-brain barrier in vivo, for example, an endfeet of glial cells (a nerve terminal connected to a dendrite of another nerve) passing through pores of an ECM condensed-and-coated membrane to come into physical contact with the vascular endothelial cells.

In addition, referring to FIG. 21B, when glial cells come into contact with vascular endothelial cells within a blood-brain barrier microfluidic chip of the disclosure to form a structure similar to an actual blood-brain barrier, through this way, it can be confirmed that a specific mRNA expression of blood-brain barrier genes known to enhance functions was significantly increased compared to single culture only in the vascular endothelial cells.

Also, referring to FIG. 21B, it can be confirmed that, when compared to an mRNA expression amount measured from a blood-brain barrier microfluidic chip manufactured by using an existing membrane having small pore size (1 □), an mRNA expression amount measured from a blood-brain barrier microfluidic chip (10 □) manufactured through the disclosure was higher.

In this way, it can be know that the functional membrane according to the disclosure and the microfluidic chip including the functional membrane may promote interaction between different cells, such as vascular endothelial cells and glial cells, more than an existing model.

### Example 5

To check whether the functional membrane of the disclosure and the microfluidic chip including the functional membrane are suitable for the development and study of metastasis models, confocal microscopy was used to observe whether the functional membrane and microfluidic chip of the disclosure allows movement of tumor cells while providing a junction site where vascular endothelial cells may sufficiently settle.

In particular, MCF-7 and MDA-MB-231 breast cancer cells were fluorescent stained with 5 μM CellTracker™ Green and then flowed to an upper channel of the blood-brain barrier microfluidic chip (Example 3) of the disclosure simulating blood vessels by using a micropipette, and after 24 hours, the channel was washed with 1×PBS to remove unattached tumor cells. Thereafter, it was confirmed whether cancer metastasized within the microfluidic chip by using a confocal microscope.

Each breast cancer cell (MCF-7, MDA-MB-231) was cultured in an incubator at 37° C. and 5% CO₂ conditions by using an RPMI-1640 medium having 10% FBS and 1% P/S added therein, a medium was changed once every 2 to 3 days, and when the breast cancer cell reached 80% or more in a culture flask, subculture was performed by using 0.25% trypsin/EDTA solution.

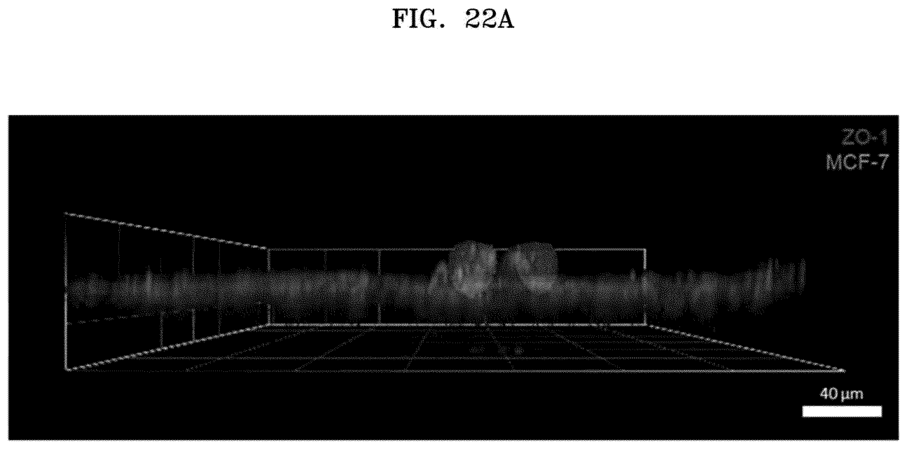

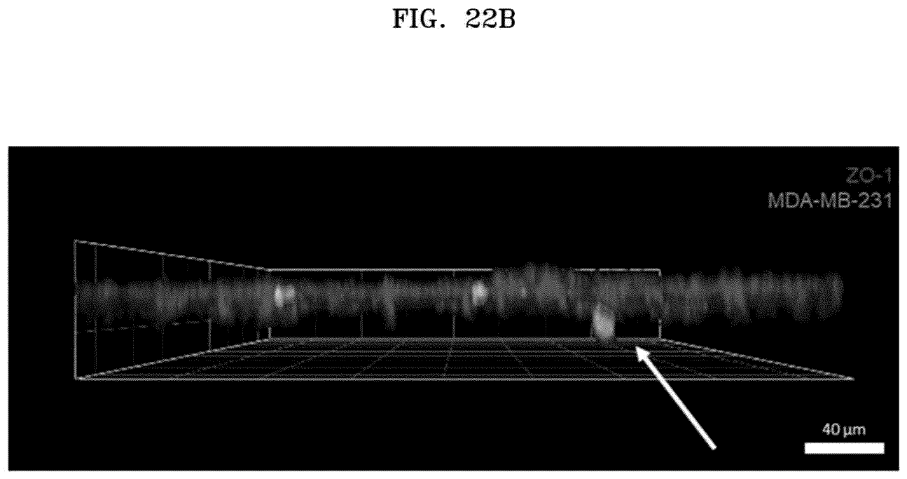

FIG. 22A shows a 3D image of a vascular endothelial cell layer and a non-metastatic breast cancer cell (MCF-7), and FIG. 22B shows a 3D image of a vascular endothelial cell layer and a metastatic breast cancer cell (MDA-MB-231).

As shown in FIGS. 22A and 22B, the MCF-7 breast cancer cells, which are poorly

metastatic, may not metastasize from an upper channel to a lower channel, and on the contrary, the MDA-MB-231 breast cancer cells, which are highly metastatic, were observed in a lower channel of the microfluidic chip by passing a vascular endothelial cell layer labeled with ZO-1 and pores (10 μm) of of a condensed-ECM-coated membrane.

Accordingly, it was confirmed that both dynamic interaction between tumor cells and the blood-brain barrier and the visual study of brain metastasis of tumor cells are possible through the functional membrane and the microfluidic chip according to the disclosure.

In this way, the membrane 300 according to an embodiment and the microfluidic chip 10 including the same may simultaneously reproduce and observe characteristics, such as blood-brain barrier formation, high cell interaction, and cancer metastasis, that may not be reproduced simultaneously in microfluidic chip models manufactured with existing commercial membrane, by performing simple ECM condensation and coating.

While the disclosure has been particularly shown and described with reference to exemplary embodiments shown in drawings, it will be understood by those of ordinary skill in the art that various changes in form and equivalent embodiments may be made from the embodiments. Accordingly, it will be understood that various changes in form and details may be made therein without departing from the spirit and scope as defined by the following claims.

Particular technical contents described in the embodiment is an example and does not limit the technical scope of the embodiment. To describe the disclosure concisely and clearly, descriptions of general techniques and configurations in the related art may be omitted. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the inventive concept unless the element is specifically described as “essential” or “critical”.

The use of the terms “a” and “an” and “the” and similar referents in the context of describing the disclosure and following claims are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Also, the steps of all methods described herein can be performed in any suitable order unless otherwise indicated herein or otherwise clearly contradicted by context. The disclosure is not limited to the described order of the steps. The use of any and all examples, or exemplary language (e.g., “such as”) provided herein, is intended merely to better illuminate the disclosure and does not pose a limitation on the scope of the disclosure unless otherwise claimed. Numerous modifications and adaptations will be readily apparent to one of ordinary skill in the art without departing from the spirit and scope.

### Industrial Applicability

The disclosure may be used in an industrial field of functional membranes and microfluidic chips.

1. A method of manufacturing a functional membrane, the method comprising:
preparing a membrane having one or more pores; and
applying a coating material on at least one surface of the membrane, and drying the coated coating material to arrange the coating material on the at least one surface of the membrane, wherein the coating material covers the one or more pores.

2. The method of claim 1, wherein the arranging of the coating material further comprises gelling the coated coating material.

3. The method of claim 1, wherein, in the arranging of the coating material, a multi-layered structure is formed by arranging the coating material multiple times.

4. The method of claim 3, wherein, in the arranging of the coating material, coating and gelling the coating material on at least one surface of the membrane and then drying and condensing the gelled coating material, coating new coating material on the condensed coating material and then gelling the coated coating material, and drying and condensing the gelled coating material is repeated.

5. The method of claim 3, wherein, in the arranging of the coating material, coating and gelling the coating material on at least one surface of the membrane and then drying and condensing the gelled coating material, coating new coating material on the condensed coating material and then drying and condensing the coated coating material is repeated.

6. The method of claim 3, wherein, in the arranging of the coating material, coating the coating material on at least one surface of the membrane and then drying and condensing the coated coating material, coating new coating material on the condensed coating material and then drying and condensing the coated coating material is repeated.

7. The method of claim 1, wherein the coating material comprises a material in a gel state or in a sol state before gelation.

8. The method of claim 1, wherein, in the arranging of the coating material, a concentration of the coating material is adjusted to adjust at least one of a thickness, density, pore size, and permeability of the coating material, which is dried and condensed.

9. A method of manufacturing a microfluidic chip, the method comprising:
manufacturing a membrane by the method according to any one of claims 1 to 8;
preparing substrates in which culture grooves are patterned; and
arranging the membrane between the substrates.

10. A method of manufacturing a microfluidic chip, the method comprising:
preparing substrates in which culture grooves are patterned;
preparing a membrane having one or more pores;
arranging the membrane on the substrates; and
arranging, on at least one surface of the membrane, a coating material covering the pores, by injecting the coating material into the culture grooves to apply the coating material on the at least one surface of the membrane and drying the coated coating material.

11. The method of claim 10, wherein the arranging of the coating material further comprises gelling the coated coating material.

12. The method of claim 10, wherein, in the arranging of the coating material, a multi-layered structure is formed by arranging the coating material multiple times.

13. The method of claim 12, wherein, in the arranging of the coating material, coating and gelling the coating material on at least one surface of the membrane and then drying and condensing the gelled coating material, coating new coating material on the condensed coating material and then gelling the coated coating material, and drying and condensing the gelled coating material is repeated.

14. The method of claim 12, wherein, in the arranging of the coating material, coating and gelling the coating material on at least one surface of the membrane and then drying and condensing the gelled coating material, coating new coating material on the condensed coating material and then drying and condensing the coated coating material is repeated.

15. The method of claim 12, wherein, in the arranging of the coating material, coating the coating material on at least one surface of the membrane and then drying and condensing the coated coating material, coating new coating material on the condensed coating material and then drying and condensing the coated coating material is repeated.

16. The method of claim 10, wherein the arranging of the coating material further comprises removing bubbles generated inside the culture grooves.

17. The method of claim 10, wherein the coating material comprises a material in a gel state or in a sol state before gelation.

18. The method of claim 10, further comprising, before the arranging of the coating material, performing pretreatment on all or part of the membrane to have a higher surface energy than the substrates.

19. The method of claim 18, wherein the membrane comprises a material capable of becoming more hydrophilic than the substrates after being subjected to the pretreatment.

20. The method of claim 18, wherein, in the performing of the pretreatment, a blocking solution is injected into the culture grooves and reacted for a predetermined period of time, and then the blocking solution is absorbed.

21. The method of claim 18, wherein, in the performing of the pretreatment, inner portions of the culture grooves are treated with plasma gas.

22. The method of claim 10, wherein the arranging of the coating material comprises adjusting a concentration of the coating material to adjust at least one of a thickness, density, pore size, and permeability of the coating material, which is dried and condensed.

23. A functional membrane comprising:
a membrane having one or more pores; and
a coating material having a multi-layered structure on the membrane and arranged on at least one surface of the membrane to cover the pores.

24. A microfluidic chip comprising:
a first substrate in which a first culture groove is patterned;
a second substrate arranged to face the first substrate and in which a second culture groove is patterned to face the first culture groove;
a membrane arranged between the first substrate and the second substrate to divide a first culture channel and a second culture channel, and having one or more pores; and
a coating material arranged on at least one surface of the membrane to cover the pores.

25. The microfluidic chip of claim 24, wherein cells injected into the microfluidic chip are cultured separately in the first culture channel and the second culture channel with the membrane therebetween, and then move to neighboring culture channels through the pores by passing through the coating material.

26. The microfluidic chip of claim 24, wherein a surface of the membrane is subjected to pretreatment to have a higher surface energy than the first substrate and the second substrate.

27. The microfluidic chip of claim 26, wherein the membrane comprises a material capable of becoming more hydrophilic than the first substrate and the second substrate after being subjected to the pretreatment.

28. The microfluidic chip of claim 26, wherein the membrane is treated with a blocking solution to have a higher surface energy than the first substrate and the second substrate.

29. The microfluidic chip of claim 26, wherein the membrane is treated with plasma gas to have a higher surface energy than the first substrate and the second substrate.
